(19) United States
(12) Patent Application Publication
(10) Pub. No.: US 2025/0345089 A1
(43) Pub. Date: Nov. 13, 2025

(54) SYSTEMS AND METHODS FOR PITCH ANGLE MOTION ABOUT A VIRTUAL CENTER

(71) Applicant: Vicarious Surgical Inc., Waltham, MA (US)

(72) Inventors: Austin Bridges, Boston, MA (US); Andrew Stein, Arlington, MA (US)

(21) Appl. No.: 18/871,265

(22) PCT Filed: Jun. 30, 2023
(86) PCT No.: PCT/US2023/026693

§ 371 Date: Dec. 3, 2024

Related U.S. Application Data
(60) Provisional application No. 63/357,891, filed on Jul. 1, 2022.

(51) Int. Cl. A61B 17/34; A61B 17/00; A61B 34/00; A61B 90/50

(52) U.S. Cl. CPC A61B 17/3403; A61B 34/71; A61B 90/50; A61B 2017/00398; A61B 2017/00407; A61B 2017/3405; A61B 2090/5025; A61B 2090/506; A61B 2090/508

(57) ABSTRACT

Systems and methods for controlling pitch motions of surgical instruments about a virtual center are provided herein. An example system includes at least one angled linkage body, at least one parallel linkage body, and a pitch housing assembly having a pitch housing, a first rotary joint having a first rotation axis, and an actuator assembly. The system further includes a second rotary joint having a second rotation axis parallel to the first rotation axis, a third rotary joint having a third rotation axis parallel to the first rotation axis, at least one angled rigid member configured to cause a rotation of the at least one parallel linkage body relative to the at least one angled linkage body about the second rotary joint, and at least one parallel rigid member configured to cause a positioning arm rotation relative to the at least one parallel linkage body about the third rotary joint.

### CROSS-REFERENCE TO RELATED APPLICATION

This application claims the benefit of priority to U.S. Provisional Patent Application No. 63/357,891 filed on Jul. 1, 2022, the entire contents of which are incorporated herein by reference.

### BACKGROUND

Some robotic surgical systems employ an external pitch control system to control a pitch of one or more supports for robotic instruments and/or camera assemblies that are inserted through a trocar or port into an internal body cavity of a subject. Such pitch control systems provide a required range of angular control or adjustment while maintaining a stationary virtual center, which reduces damage to the patient due to pressures and stresses from movement of the trocar or port. Some conventional pitch systems including a curvilinear rail for pitch control are stable and maintain a selected pitch even when contacted by an external upward force, but are relatively heavy and require a relatively large amount of space near the patient, which can limit physical access to the patient and reduce mobility of the system.

Some conventional pitch systems employ linkages for pitch control. For example, some conventional pitch systems employing linkages for pitch control use overlapping metal bands, as well as belts and pulleys to transmit torque from one linkage to another, resulting in a relatively wide range pitch control system. Some conventional pitch systems employing linkages for pitch control rely on computer controlled coordinated motion of the various linkages to enforce the stationary virtual center, which increases a risk of failure relative to mechanically enforcing the stationary virtual center.

### SUMMARY

The present disclosure provides systems and methods for controlling pitch motions of surgical instruments. A pitch system for controlling a pitch orientation of one or more surgical instruments relative to a virtual center is provided herein in accordance with some embodiments. The pitch system includes at least one angled linkage body having a proximal end, a distal end, and a first linkage body axis at an acute angle with respect to a yaw axis of the pitch system during use, at least one parallel linkage body having a proximal end, a distal end, and a second linkage body axis parallel to the yaw axis of the pitch system during use, and a pitch housing assembly having a first end configured to be connected with, configured to be connected to, connected with, or connected to a yaw system defining the yaw axis. The pitch housing assembly includes a pitch housing, a first rotary joint having a first rotational axis perpendicular to and intersecting the yaw axis, and actuator assembly configured to drive a rotation of the at least one angled linkage body relative to the pitch housing about the first rotary joint causing an angled linkage body rotation.

The pitch system further includes a second rotary joint having a second rotation axis parallel to the first rotation axis. The proximal end of the at least one angled linkage body is rotationally coupled to the pitch housing at the first rotary joint and a proximal end of the at least one parallel linkage body is rotationally coupled to the at least one angled linkage body at the second rotary joint.

The pitch system further includes a third rotary joint having a third rotation axis parallel to the first rotation axis, at least one angled rigid member configured to cause a parallel linkage body rotation of the at least one parallel linkage body relative to the at least one angled linkage body about the second rotary joint due to the angled linkage body rotation. The proximal end of the at least one angled rigid member is rotationally coupled to the pitch housing at a first pivot axis parallel to and offset from the first rotation axis, and a distal end of the at least one angled rigid member is rotationally coupled to the at least one parallel linkage body at a second pivot axis parallel to and offset from the second rotation axis. The proximal end of the at least one parallel linkage body is rotationally coupled to the distal end of the at least one angled linkage body at the second rotary joint, and the distal end of the at least one parallel linkage body is rotationally coupled to a positioning arm or a mounting (e.g., a mounting plate) for a positioning arm at the third rotary joint.

As used throughout, references “to a positioning arm” or “the positioning arm” should be interpreted as references to “a positioning arm or a mounting for the positioning arm” or “the positioning arm or the mounting for the positioning arm,” where appropriate.

The pitch system further includes at least one parallel rigid member configured to cause a positioning arm rotation of the positioning arm relative to the at least one parallel linkage body about the third rotary joint due to the parallel linkage body rotation. The proximal end of the at least one parallel rigid member is rotationally coupled with the at least one angled linkage body at a third pivot axis parallel to and offset from the second rotary joint, and a distal end of the at least one parallel rigid member is rotationally coupled with the positioning arm for the positioning arm at a fourth pivot axis offset from and parallel to the third rotary joint.

When in use, an intersection point at an intersection of the yaw axis and the pitch axis is the virtual center. When in use, the pitch housing assembly, the at least one angled rigid member, and the at least one parallel rigid member are configured to constrain motion of the at least one angled linkage body, the at least one parallel linkage body, and the positioning arm to maintain an orientation of the second linkage body axis parallel to the yaw axis and to maintain an orientation of the first linkage body axis body parallel to a line perpendicular to the third rotational axis extending from the virtual center to the third rotational axis during rotation of the at least one angled linkage body relative to the pitch housing.

In one embodiment, the orientation of the first linkage body axis is defined as an orientation of a first line perpendicular to the second rotation axis and extending from the second rotation axis at the second rotary joint though the first rotations axis and intersecting the yaw axis. The orientation of the at least one second linkage body axis is defined as an orientation of a second line perpendicular to and extending from the third rotation axis at the third rotary joint to the second rotation axis and intersecting with the first line.

In one embodiment, the at least one angled linkage body comprises a first angled linkage side plate and a second angled linkage side plate.

In one embodiment, the at least one angled rigid member comprises a first side angled rigid member and a second side angled rigid member.

In one embodiment, the at least one parallel linkage body comprises a first parallel linkage side plate and a second parallel linkage side plate.

In one embodiment, the at least one parallel rigid member includes a central rigid member.

In one embodiment, the at least one parallel linkage body comprises a first parallel linkage side plate and a second parallel linkage side plate each having a proximal end and a distal end. The at least one angled rigid member comprises a first side angled rigid member and a second side angled rigid member each having a proximal end and a distal end. The distal end of the first side angled rigid member is rotationally connected to the proximal end of the first parallel linkage side plate at the second pivot axis, and the distal end of the second side angled rigid member is rotationally connected to the proximal end of the second parallel linkage side plate at the second pivot axis.

In one embodiment, the at least one angled linkage body comprises a first angled linkage side plate and a second angled linkage side plate each having a proximal end and a distal end. The second rotary joint includes a second rotary joint shaft rotationally locked to the at least one angled linkage body. The at least one parallel rigid member includes a central parallel rigid member having a proximal end and a distal end. The pitch system also includes a first mounting bracket including a first axle shaft. The first mounting bracket is attached to and rotationally locked to the second rotary joint shaft. The first mounting bracket rotatably connects with the proximal end of the central rigid member at the third pivot axis via the first axle shaft. The pitch system also includes a second mounting bracket including a second axle shaft. The second mounting bracket is affixed to or connected to the mounting for the positioning arm or to the positioning arm. The second mounting bracket is rotatably connected with the distal end of the central parallel rigid member at the fourth pivot axis via the second axle shaft.

In one embodiment, the actuator assembly comprises at least one motor subassembly configured to drive an output rotation about a drive axis relative to the pitch housing and at least one coupling configured to couple a rotation of the at least one angled linkage side plate about the first rotary joint with the output rotation about the drive axis.

In one embodiment, the at least one motor subassembly comprises a motor pulley.

In one embodiment, the at least one motor subassembly comprises a motor, an encoder, and a gearhead.

In one embodiment, the motor, the encoder and the gearhead are disposed within a motor pulley.

In one embodiment, the actuator assembly further comprises at least one output pulley rotationally locked to the rotary shaft of the first rotary joint.

In one embodiment, the at least one coupling comprises at least one drive tape affixed to the motor pulley and to the output pulley.

In one embodiment, the rotary shaft of the first rotary joint is rotationally locked to the proximal end of the at least one angled linkage body.

In one embodiment, the rotary shaft of the first rotary joint is not physically rotationally locked to pitch housing.

In one embodiment, the actuator assembly further comprises a braking system configured for braking of the rotary shaft of the first rotary joint relative to the pitch housing.

In one embodiment, the braking system comprises a brake stator fixed to the pitch housing and a brake rotor fixed to the rotary shaft of the first rotary joint.

In one embodiment, the system further comprises a secondary braking system comprising a pawl and ratchet gear configured to prevent the positioning arm from rotating in at least one direction of rotation.

In one embodiment, the pawl is configured to disengage from the ratchet gear when power is supplied to a solenoid actuator, and wherein the pawl is configured to reengage with the ratchet gear via a spring when power is removed from the solenoid actuator.

In one embodiment, the first rotary joint comprises a first rotary shaft rotationally locked to the at least one angled linkage body. The pitch housing comprises a first pitched housing side plate and a second pitched housing side plate, the first rotary shaft not physically locked to the first pitched housing side plate or the second pitched housing side plate.

In one embodiment, the pitch housing assembly further comprises one or more springs configured to offset torsional moment created by weights of downstream components.

In one embodiment, the second rotary joint comprises a second rotary shaft rotationally locked to the at least one parallel linkage body. The rotation of the second rotary shaft is locked to that of the at least angled linkage body.

In one embodiment, the third rotary joint comprises a third rotary shaft rotationally locked to the positioning arm. The rotation of the third rotary shaft is locked to the second rotary shaft via the at least parallel one parallel linkage body.

In one embodiment, the third rotary joint comprises a third rotary shaft rotationally locked to the at least one parallel linkage body, and rotatably connected to the positioning arm or the mounting of the positioning arm, wherein rotation of the positioning arm about the third rotary axis is locked to the second rotary shaft via the at least one parallel linkage body.

In one embodiment, the first rotary joint, the second rotary joint, or the third rotary joint comprises one or more shielded ball bearings, one or more preloaded bearings, or one or more bearings in a back-to-back arrangement.

In one embodiment, the pitch axis extends normal to the insertion axis of the positioning arm and intersects the cannula axis of a trocar.

When in use, an intersection point at an intersection of the yaw axis and the pitch axis is the virtual center.

When in use, the pitch housing assembly, the at least one angled rigid member, and the at least one parallel rigid member are configured to constrain motion of the at least one angled linkage body, the at least one parallel linkage body, and the positioning arm to maintain an orientation of the second linkage body axis parallel to the yaw axis and to maintain an orientation of the first linkage body axis body parallel to a line perpendicular to the third rotational axis extending from the virtual center to the third rotational axis during rotation of the at least one angled linkage body relative to the pitch housing in accordance with some embodiments.

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. These and other features and advantages of the present invention will be more fully understood by reference to the following detailed description in conjunction with the attached drawings in which like reference numerals refer to like elements throughout the different views.

FIG. 1 schematically depicts a surgical robotic system in accordance with some embodiments.

FIG. 2A is a perspective view of a patient cart including a robotic support system coupled to a robotic subsystem of the surgical robotic system in accordance with some embodiments.

FIG. 2B is a perspective view of an example operator console of a surgical robotic system of the present disclosure in accordance with some embodiments.

FIG. 3A schematically depicts a side view of a surgical robotic system performing a surgery within an internal cavity of a subject in accordance with some embodiments.

FIG. 3B schematically depicts a top view of the surgical robotic system performing the surgery within the internal cavity of the subject of FIG. 3A in accordance with some embodiments.

FIG. 4A is a perspective view of a single robotic arm subsystem in accordance with some embodiments.

FIG. 4B is a perspective side view of a single robotic arm of the single robotic arm subsystem of FIG. 4A in accordance with some embodiments.

FIG. 5 is a perspective front view of a camera assembly and a robotic arm assembly in accordance with some embodiments.

FIG. 6 is a side view of a pitch control system mounted on a mobile patient cart, and a positioning arm including insertion rails, instrument drives and cassettes and a trocar mount connected to the pitch control system in accordance with some embodiments.

FIG. 7 is a perspective view of a pitch system and a positioning arm in accordance with some embodiments.

FIG. 8A depicts an example pitching housing assembly in accordance with some embodiments.

FIG. 8B depicts a perspective view of a pair of angled linkage bodies and a pair of angled rigid members in accordance with some embodiments.

FIG. 8C depicts a perspective view of a pair of parallel linkage bodies and a parallel rigid member in accordance with some embodiments.

FIG. 8D depicts a perspective view of a positioning arm including insertion rails, a camera drive and cassette, instrument drives and cassettes and a trocar mount in accordance with some embodiments.

FIG. 9 is a side view of a pitch system and positioning arm in accordance with some embodiments.

FIG. 10A is a side view illustrating a pitch system with an insertion axis perpendicular to a yaw axis and with an insertion angle of zero degrees relative to a horizontal line passing through a virtual center in accordance with some embodiments.

FIG. 10B is a side view of the pitch system of FIG. 10A adjusted to pitch down the insertion axis for an angle of negative 30 degrees relative to the horizontal line passing through the virtual center in accordance with some embodiments.

FIG. 10C schematically depicts an is a side view of the pitch system of FIG. 10A adjusted to pitch up the insertion axis for an angle of positive 15 degrees relative to the horizontal line passing through the virtual center in accordance with some embodiments.

FIG. 11A is a side view of a patient cart, pitch system and positioning arm with a motor unit of the positioning arm in a retracted position in accordance with some embodiments.

FIG. 11B is a side view of a patient cart, pitch system and positioning arm of FIG. 11A with the motor unit in an insertion position in accordance with some embodiments.

FIG. 12A is a perspective view of a pitch housing assembly in accordance with some embodiments.

FIG. 12B is a perspective view of the pitch housing assembly of FIG. 12A with one pitch housing side plate shown as translucent and a pitch housing top plate shown as transparent for illustrative purposes in accordance with some embodiments.

FIG. 13A is a perspective view of a motor subassembly of a pitch housing assembly in accordance with some embodiments.

FIG. 13B is a perspective view of a motor subassembly in accordance with some embodiments.

FIG. 13C is a cross-sectional view of the motor subassembly of FIG. 13B in accordance with some embodiments.

FIG. 14A is a perspective view of distal portion of a pitch subassembly including a motor pulley and an output pulley connected by drive tapes in accordance with some embodiments.

FIG. 14B is a perspective view of the output pulley of FIG. 14A in accordance with some embodiments.

FIG. 15 is a cross-sectional view of a portion of the pitch system taken through the first rotary joint and including the pitch housing assembly, the angled linkage side plates, and angled rigid members in accordance with some embodiments.

FIG. 16A is a perspective view of a pitch housing assembly including extension springs for counterbalance in accordance with some embodiments.

FIG. 16B is a detail of FIG. 16A including a middle extension spring connected to interior facing ends of output pulleys via an attachment post in accordance with some embodiments.

FIG. 16C is a detail of FIG. 16A including side extension springs connected to an exterior facing end of an output pulley via attachment posts in accordance with some embodiments.

FIG. 17A is a detail of FIG. 8B depicting a distal portion of a first side angled rigid member and first angled linkage side plate in accordance with some embodiments.

FIG. 17B is a front view of a distal end of the one of the angled rigid members of FIG. 8B in accordance with some embodiments.

FIG. 17C is a detail of FIG. 8B depicting a proximal end of a first side angled rigid member and first angled linkage side plate in accordance with some embodiments.

FIG. 17D is a cross-sectional view of the proximal end of the angled rigid member of FIG. 17C rotationally coupled to an offset plate configured to attached to the pitch housing in accordance with some embodiments.

FIG. 18 is a perspective view of a parallel linkage body, a parallel rigid member, a second rotary joint a third rotary joint and a mounting bracket to be mounted to a positioning arm in accordance with some embodiments and identifies cross-sections for the cross-sectional views of FIGS. 19, 20A, 21 and 23.

FIG. 19 is a cross-sectional view of the second rotary joint, angled linkage side plates rotationally locked to a shaft of the second rotary joint, parallel linkage side plates, angled rigid members rotationally connected to the parallel linkage side plates at a third pivot axis, and an first mounting bracket rotationally locked to the shaft of the second rotary joint in accordance with some embodiments.

FIG. 20A is a cross-sectional view taken through the third pivot axis of a proximal end of a parallel rigid member and first mounting bracket in accordance with some embodiments.

FIG. 20B is a perspective view of the proximal end of the parallel rigid member and the first mounting bracket of FIG. 20A with the first mounting bracket depicted as translucent for illustrative purposes.

FIG. 20C is a different perspective view of the proximal end of the parallel rigid member and the first mounting bracket of FIG. 20A in accordance with some embodiments.

FIG. 21 is a cross-sectional view taken through the fourth pivot axis of a distal end of a parallel rigid member and second mounting bracket in accordance with some embodiments.

FIG. 22A is a perspective view of a shaft subassembly of a third rotary joint in accordance with some embodiments.

FIG. 22B is a cross-sectional view taken through the third rotary axis of the shaft subassembly of FIG. 22A in accordance with some embodiments.

FIG. 23 is a cross-sectional view of the shaft subassembly of FIG. 22A rotationally coupled to distal ends of parallel linkage side plates by the third rotary joint and attached to a mounting (e.g., a mounting plate) of a positioning arm in accordance with some embodiments.

FIG. 24A is a top perspective view of another example pitch system in accordance with some embodiments.

FIG. 24B is a different perspective view of the pitch system of FIG. 24 in accordance with some embodiments.

FIG. 25A is a perspective view of another example pitch system in accordance with some embodiments.

FIG. 25B is a side view of the pitch system of FIG. 25A in accordance with some embodiments.

FIG. 26A is a side view of a pitch system adjusted to pitch up an insertion axis for an angle of positive 30 degrees relative to a horizontal line passing through a virtual center in accordance with some embodiments.

FIG. 26B is a side view of the pitch system adjusted to pitch down the insertion axis for an angle of negative 20 degrees relative to the horizontal line passing through the virtual center in accordance with some embodiments.

FIG. 27A is a perspective view from a first side of an angled linkage body in accordance with some embodiments.

FIG. 27B is a perspective view from a second side of the angled linkage body of FIG. 27A in accordance with some embodiments.

FIG. 28A is a perspective view of an angled rigid member in accordance with some embodiments.

FIG. 28B is a cross-sectional view of a proximal end of the angled rigid member of FIG. 28A in accordance with some embodiments.

FIG. 28C is a cross-sectional view of a distal end of the angled rigid member of FIG. 28A in accordance with some embodiments.

FIG. 29A is a perspective view of an angled linkage body rotationally coupled to a parallel linkage body in accordance with some embodiments.

FIG. 29B is a perspective view from a first side of a connection portion connecting the angled linkage body with the parallel linkage body of FIG. 29A in accordance with some embodiments.

FIG. 29C is a perspective view from a second side of the connection portion of FIG. 29B in accordance with some embodiments.

FIG. 30A is a perspective view of a parallel linkage body rotationally coupled to a second rotary joint and a third rotary joint in accordance with some embodiments.

FIG. 30B is a cross-sectional view of the parallel linkage body of FIG. 30A in accordance with some embodiments.

FIG. 30C is a rear cross-sectional view of a second rotary joint coupled to a proximal end of the parallel linkage body of FIG. 30A in accordance with some embodiments.

FIG. 30D is a bottom cross-sectional view of the second rotary joint of FIG. 30C in accordance with some embodiments.

FIG. 30E is a cross-sectional view of a third rotary joint coupled to a distal end of the parallel linkage body of FIG. 30A in accordance with some embodiments.

FIG. 30F is a cross-sectional view of the third rotary joint of FIG. 30E further coupled to a third joint bracket in accordance with some embodiments.

FIG. 31A is a side view of a parallel rigid member in accordance with some embodiments.

FIG. 31B is a perspective view of the parallel rigid member of FIG. 31A in accordance with some embodiments.

FIG. 31C is a cross-sectional view of a proximal end of the parallel rigid member of FIG. 31A in accordance with some embodiments.

FIG. 31D is a cross-sectional view of the distal end of the parallel rigid member of FIG. 31A in accordance with some embodiments.

FIG. 32A is a perspective view of a parallel linkage body coupled to a parallel rigid member in accordance with some embodiments.

FIG. 32B is a perspective view of the parallel linkage body and the parallel rigid member of FIG. 33A coupled to a mounting plate in accordance with some embodiments.

FIG. 32C is a perspective view from bottom of a bottom portion of the parallel linkage body and the parallel rigid member of FIG. 32A further coupled to a third joint bracket in accordance with some embodiments.

FIG. 32D is a perspective view from bottom of a mounting plate accordance with some embodiments.

FIG. 33A is a perspective view from left side of a pitch housing assembly having an actuator assembly connecting to an angled linkage body and an angled rigid member in accordance with some embodiments.

FIG. 33B is a cross-sectional view of the pitch housing assembly in FIG. 33A in accordance with some embodiments.

FIG. 34A is a perspective view from right side of a pitch housing assembly of FIG. 33A illustrating a braking system in accordance with some embodiments.

FIG. 34B is a cross-sectional view of a primary brake in accordance with some embodiments.

FIG. 34C is a side view of a secondary brake in accordance with some embodiments.

FIG. 34D is a front view of the secondary brake of FIG. 34C in accordance with some embodiments.

### DETAILED DESCRIPTION OF THE INVENTION

While various embodiments of the invention have been shown and described herein, it will be obvious to those skilled in the art that such embodiments are provided by way of example only. Numerous variations, changes, and substitutions may occur to those skilled in the art without departing from the invention. It may be understood that various alternatives to the embodiments of the invention described herein may be employed.

As used in the specification and claims, the singular form “a,” “an,” and “the” include plural references unless the context clearly dictates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items.

Unless specifically stated or obvious from context, as used herein, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

Systems and methods for pitch angle adjustment about a virtual center are described and depicted herein. As described above, some robotic surgical systems employ an external pitch control system to control a pitch of one or more supports for robotic instruments and/or camera assemblies that are inserted through a trocar or port into an internal body cavity of a subject. A pitch control system can provide required range of angular control or adjustment while maintaining a stationary virtual center.

Some embodiments of a pitch control system provided include linkages connecting a pitch housing to a positioning arm. The linkages, which may also be referred to as couplings herein, are rotatably connected to each other and to the pitch housing or to the positioning arm by rotary joints having rotation axes. Rigid members rotatably couple each linkage to the pitch housing or to the positioning arm at pivot axes that are offset from the rotation axis of the rotary joints. The rigid members cause a rotation of a next linkage due to rotation of a current linkage (e.g., rotation of a first linkage driven by a motor causing a rotation of a second linkage), and cause a rotation of the positioning arm due to rotation of a last linkage (e.g., where there are two linkages, cause a rotation of the positioning arm driven by a rotation of the second linkage). Interaction of the rigid members, the pitch housing, the linkages, and the positioning arm mechanically enforces a stationary location of a virtual center in the pitch control system. In some embodiments, bearings are employed at rotational joints and at the pivot axes for the rigid members.

As explained above, mechanically enforcing a stationary location of a virtual center in the pitch control system may be more reliable and less prone to failure than other methods and mechanisms for enforcing a stationary location of a virtual center, such as software or active controls.

Systems and methods described herein employing linkages or linkage bodies and rigid members for pitch control may have reduced size and weight compared to systems for pitch control employing a curvilinear rail, and increased stiffness compared to systems for pitch control employing linkages and belts or pulleys to transmit rotation of one linkage to another.

In some embodiments, systems described herein include only two linkages, which may alternatively be described as two link stages or two couplings. The two linkages or two link stages include a first linkage or first link stage including at least one first linkage body that has an angled orientation with respect to a yaw axis and that rotatably connects with a pitch housing, and a second linkage or second link stage including at least one second linkage body that has a parallel orientation with respect to a yaw axis and that rotatably connects with a positioning arm. In some embodiments, employing only two linkages or only two link stages corresponds to a reduced size and weight for the pitch system. Further, in some embodiments employing only two linkages, a complexity of the system is reduced, thereby reducing potential sources of error or failure.

In some embodiments, the system includes only one rigid member per coupling or per linkage stage for one or both of the linkage stages.

Systems and methods for controlling pitch motions of surgical instruments about a virtual center are provided herein. An example system includes at least one angled linkage body, at least one parallel linkage body, and a pitch housing assembly having a pitch housing, a first rotary joint having a first rotation axis, and an actuator assembly. The actuator assembly can include at least one, motor, at least one gearhead, at least one braking device, at least one device for measuring relative or absolute angular position (e.g. optical or magnetic induction encoder), at least one belt spanning at least two pulleys, other means of generating rotational motion, or some combination thereof. The system further includes a second rotary joint having a second rotation axis parallel to the first rotation axis, a third rotary joint having a third rotation axis parallel to the first rotation axis, at least one angled rigid member configured to cause a rotation of the at least one parallel linkage body relative to the at least one angled linkage body about the second rotary joint, and at least one parallel rigid member configured to cause a positioning arm rotation relative to the at least one parallel linkage body about the third rotary joint.

Some embodiments of a pitch control system include a pitch housing holding a driving system (e.g., including a motor and speed reduction). An output axis from the driving system is coincident and rotationally coupled with first linkage that includes one or more linkage bodies at a first rotary joint having a first rotation axis, which is the output axis. The pitch housing remains stationary relative to the rest of the pitch subsystem and is fixed to a housing of the motor such that motion about the output axis from the driving system, which is the first rotation axis, is relative to the pitch housing. The at least one first rigid member is rotationally coupled with the pitch housing at first pivot axis a radial distance “r” from the first rotation axis in some embodiments. In some embodiments, only one first rigid member is rotationally coupled with the pitch housing at the pivot axis a radial distance “r” from the first rotation axis. For example, a first rigid member could be connected to the pitch housing via a hinge joint with a clevis pin, such that the first rigid member is allowed to rotate about the pin. The axis of the pin itself, corresponding to the first pivot axis, is not allowed to rotate about the first rotation axis. The other end of the first rigid member is rotationally coupled with a second linkage, specifically a second linkage body of a second linkage, in a similar manner to allow the first rigid member to rotate about a connection axis (e.g., of another clevis pin) that is a second pivot axis. The connection axis can be the same distance “r” from a second rotation axis of a second rotary joint the first and second linkages. The center distance between the first pivot axis and the second pivot axis can be approximately equal in length to the center distance between the first rotary axis and the second rotary axis. As the first linkage is rotated by the driving system, the first rigid member effectively rotates relative to the first linkage in the opposite direction. Any angular displacement of the first linkage creates an equal but opposite angular displacement of the second linkage, so that the second linkage always remains parallel to the pitch housing.

A second rigid member, or at least one second rigid member is rotationally coupled to the first linkage, at a third pivot axis that is a distance “r” from the second rotation axis of the second rotary joint connecting the first and second linkages. The other end of the second rigid member is rotationally coupled to a third linkage (or positioning arm), at a fourth pivot axis that is a distance “r” from a third rotation axis of a third rotation joint connecting the second and third linkages or connecting the second linkage and a positioning arm. The third linkage remains parallel to the insertion axis, which passes through the virtual center. As the first linkage is rotated by the driving system, the second rigid member is permitted to rotate about its first connection axis, but this axis does not rotate relative to the first linkage. The other end of the second rigid member is permitted to rotate about its second connection axis, but again this axis does not rotate relative the third linkage. The center distance between the third pivot axis and the fourth pivot axis can be approximately equal in length to the center distance between the second rotary axis and the third rotary axis. Therefore, any rotation of the first linkage creates an equal rotation in the same direction of the third linkage or of the positioning arm.

The first linkage is angularly offset from the insertion axis. If the first linkage is in-plane with the insertion axis and the yaw axis, then the offset angle with respect to a horizontal line can be less than 90 degrees minus the desired limit in the negative pitch direction (e.g., pitching down the insertion axis for an angle of negative 30 degrees or other suitable degrees relative to a horizontal line passing through the virtual center), and the output axis of the driving system can be sufficiently below a yaw actuator to provide clearance for the first linkage in some embodiments. If the first linkage is or is sufficiently out-of-plane with the insertion axis and the yaw axis, then this interference could be avoided entirely. The positioning arm is at a “zero” degree pitch angle when the first linkage is at its nominal offset angle in some embodiments. The second linkage is planarly offset from the first linkage, but its midplane could potentially be coincident with the midplane of the positioning arm (if only one is used) in some embodiments. In some embodiments, a linkage may include two linkage bodies instead of one linkage body to reduce tension in the rigid members.

In some embodiments, interference could be further reduced by having the output axis of the driving system be offset in the direction of the insertion axis, such that it no longer intersects the yaw axis.

Some systems enable generation of sufficient torque to rotate a positioned arm at a desired angular velocity and acceleration while providing a sufficient range of angular displacement of a positioning arm. In some embodiments, at least one rotational actuator is concentric with the output axis of the driving system, which itself is parallel to the pitch axis and passes through the yaw axis (as described above). The at least one rotational actuator could include any suitable combination of one motor, at least one gearhead, at least one braking device, at least one device for measuring relative or absolute angular position (e.g., optical or magnetic induction encoder), at least one belt spanning at least two pulleys, or any other means of generating rotational motion. In some embodiments, at least one output pulley is concentric with the output axis of the driving system, which itself is parallel to the pitch axis and passes through the yaw axis (as described above). In some embodiments, two non-continuous belts are rigidly adhered to the outside of the pulley, such as through rivets or screws. The two belts, when placed in tension, cause the output pulley to rotate in opposite directions. The other ends of both belts are rigidly adhered to the outside of a smaller driving pulley, such that the rotation of the driving pulley in one direction causes tension in one of the belts, and rotation in the opposite direction causes tension in the other belt. In some embodiments, this pattern could be repeated to reduce the tension in each belt, so long as all of the driven pulleys are concentric, and all of the driving pulleys are concentric. In some embodiments, a number of pulleys is doubled so that there are two driven pulleys (e.g., output pulleys), two driving pulleys (e.g., motor pulleys), and a total of four belts. The driving axis is coincident with the output axis of a preliminary drive system (e.g., a motor and gearbox).

Some advantages of embodiments employing this tape belt type of system for driving an output pulley over a conventional continuous belt system, is that metal belts are used, which are stronger and stiffer than polymers such as polyurethane. Because the belts are non-continuous, they are rigidly adhered to the outside of each pulley. If the belts were co-planar, this would limit the amount of rotation that the system would be able to achieve. This set-up avoids this problem by separating the belt planes from one another. This allows for almost 360 degrees of rotation (e.g., at least 340 degrees of rotation). Further, the system creates a near-zero backlash condition, as the belts are always in tension.

The speed reduction of the pulley system, created by the diametral ratio between the driving motor input pulley and the output pulleys, is equivalent to the angular displacement reduction. An exactly 360-degree rotation is not possible in this configuration. However, for example, 45 degrees of output rotation is achievable with a speed ratio less than 8:1, given the input rotation is less than 360 degrees, to provide room for attachment endplates for the belts.

Some embodiments include a braking system coaxial with the output axis, and connecting the first linkage to the pitch housing, instead of at the motor. Because the linkages are rotationally coupled through rigid members, if the brake has enough torque capacity to counter-act the static load of the system, it prevents all rotation.

Some embodiments include compression or extension springs to apply opposing torque to the first linkage. These springs substantially reduce the required torque output from the drive system, and the required holding torque of the brake, which may reduce a size of the pitch housing and the system.

Some embodiments include a secondary braking mechanism for redundancy and safety. The secondary braking mechanism can include at least one actuated pawl and ratchet gear. The at least one pawl can be actuated linearly or rotationally about its intended pivot point. In the case of linear actuation, a slotted pawl mount and shoulder bolt can be used to create rotation of the pawl about its pivot point through linear actuation. The actuation can be generated electromechanically (e.g., via a solenoid actuator). When the electromechanical actuator is powered off, gravity and/or a return spring can be employed to engage the pawl with the ratchet gear, preventing rotation of the pitch actuator. When the electromechanical actuator is powered on, the force or torque generated by the actuator overcomes the forces of gravity and/or return spring to disengage the pawl from the ratchet gear, permitting rotation of the pitch actuator.

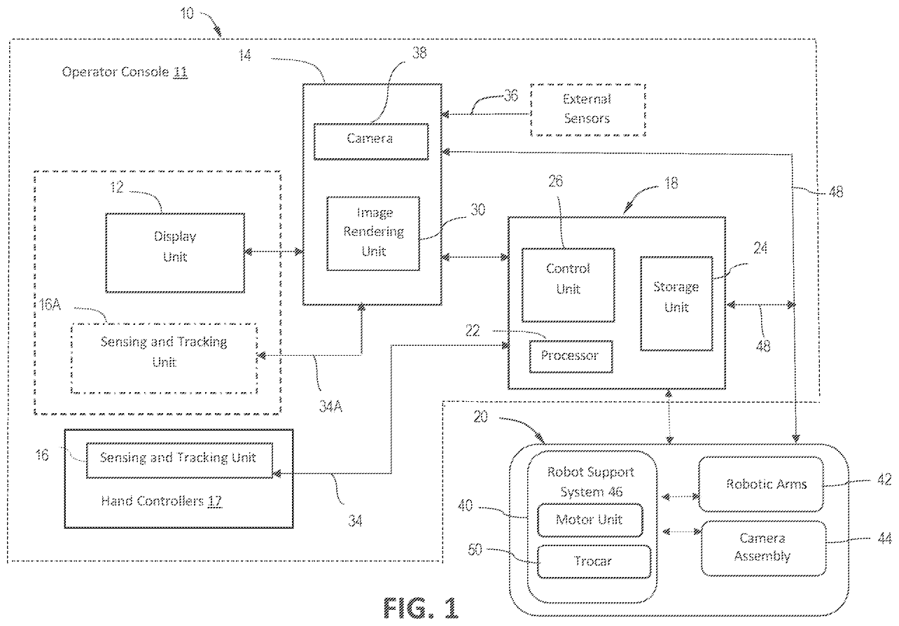

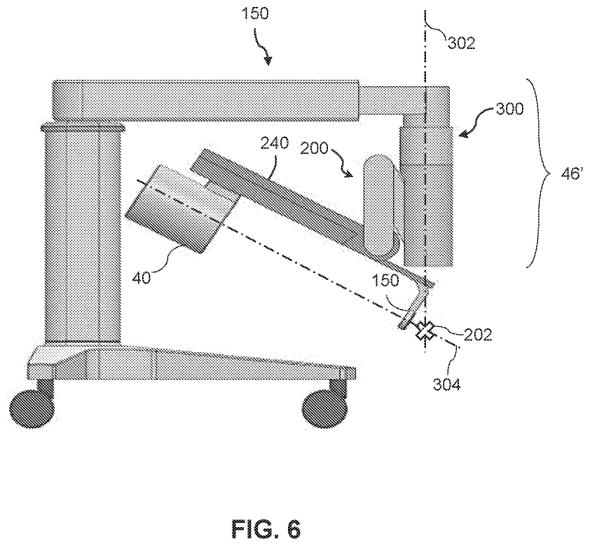

Prior to providing additional specific description of the pitch system with respect to FIGS. 6-35, a surgical robotic system in which the pitch system could be employed is described below with respect to FIGS. 1-5.

### Surgical Robotic Systems

Some embodiments may be employed with a surgical robotic system. A system for robotic surgery may include a robotic subsystem that includes a surgical robotic unit that can be inserted into a patient via a trocar through a single incision point or site. The robotic unit is small enough to be deployed in vivo at the surgical site and is sufficiently maneuverable when inserted to be able to move within the body to perform various surgical procedures at multiple different points or sites. The surgical robotic unit includes multiple separate robotic arms that are deployable within the patient along different or separate axes. Further, a surgical camera assembly can also be deployed along a separate axis. Thus, the surgical robotic unit employs multiple different components, such as a pair of robotic arms and a surgical or robotic camera assembly, each of which are deployable along different axes and are separately manipulatable, maneuverable, and movable. The robotic arms and the camera assembly that are disposable along separate and manipulatable axes is referred to herein as the Split Arm (SA) architecture. The SA architecture is designed to simplify and increase efficiency of the insertion of robotic surgical instruments through a single trocar at a single insertion site, while concomitantly assisting with deployment of the surgical instruments into a surgical ready state as well as the subsequent removal of the surgical instruments through the trocar. By way of example, a surgical instrument can be inserted through the trocar to access and perform an operation in vivo in the abdominal cavity of a patient. In some embodiments, various surgical instruments may be used or employed, including but not limited to robotic surgical instruments, as well as other surgical instruments known in the art.

The systems, devices, and methods disclosed herein can be incorporated into and/or used with a robotic surgical device and associated system disclosed for example in U.S. Pat. No. 10,285,765 and in PCT patent application Serial No. PCT/US2020/39203, and/or with the camera assembly and system disclosed in United States Publication No. 2019/0076199,and/or the systems and methods of exchanging surgical tools in an implantable surgical robotic system disclosed in PCT patent application Serial No. PCT/US2021/058820, where the content and teachings of all of the foregoing patents, patent applications and publications are incorporated herein by reference herein in their entirety. The surgical robotic unit that forms part of the present invention can form part of a surgical robotic system that includes a surgeon workstation that includes appropriate sensors and displays, and a robot support system (RSS) for interacting with and supporting the robotic subsystem of the present invention in some embodiments. The robotic subsystem includes a motor unit and a surgical robotic unit that includes one or more robotic arms and one or more camera assemblies in some embodiments. The robotic arms and camera assembly can form part of a single support axis robotic system, can form part of the split arm (SA) architecture robotic system, or can have another arrangement. The robot support system can provide multiple degrees of freedom such that the robotic unit can be maneuvered within the patient into a single position or multiple different positions. In one embodiment, the robot support system can be directly mounted to a surgical table or to the floor or ceiling within an operating room. In another embodiment, the mounting is achieved by various fastening means, including but not limited to, clamps, screws, or a combination thereof. In other embodiments, the structure may be free standing. The robot support system can mount an actuator assembly that is coupled to the surgical robotic unit, which includes the robotic arms and the camera assembly. The actuator assembly can include gears, motors, drivetrains, electronics, and the like, for powering the components of the surgical robotic unit.

The robotic arms and the camera assembly are capable of multiple degrees of freedom of movement. According to some embodiments, when the robotic arms and the camera assembly are inserted into a patient through the trocar, they are capable of movement in at least the axial, yaw, pitch, and roll directions. The robotic arms are designed to incorporate and employ a multi-degree of freedom of movement robotic arm with an end effector mounted at a distal end thereof that corresponds to a wrist area or joint of the user. In other embodiments, the working end (e.g., the end effector end) of the robotic arm is designed to incorporate and use or employ other robotic surgical instruments, such as for example the surgical instruments set forth in U.S. Publ. No. 2018/0221102, the entire contents of which are herein incorporated by reference.

Turning to the drawings, FIG. 1 is a schematic illustration of an example surgical robotic system 10 in which aspects of the present disclosure can be employed in accordance with some embodiments of the present disclosure. The surgical robotic system 10 includes an operator console 11 and a robotic subsystem 20 in accordance with some embodiments.

The operator console 11 includes a display device or unit 12, an image computing unit 14, which may be a virtual reality (VR) computing unit, hand controllers 17 having a sensing and tracking unit 16, and a computing unit 18.

The display unit 12 may be any selected type of display for displaying information, images or video generated by the image computing unit 14, the computing unit 18, and/or the robotic subsystem 20. The display unit 12 can include or form part of, for example, a head-mounted display (HMD), an augmented reality (AR) display (e.g., an AR display, or AR glasses in combination with a screen or display), a screen or a display, a two-dimensional (2D) screen or display, a three-dimensional (3D) screen or display, and the like. The display unit 12 can also include an optional sensing and tracking unit 16A. In some embodiments, the display unit 12 can include an image display for outputting an image from a camera assembly 44 of the robotic subsystem 20.

In some embodiments, if the display unit 12 includes an HMD device, an AR device that senses head position, or another device that employs an associated sensing and tracking unit 16A, the HMD device or head tracking device generates tracking and position data 34A that is received and processed by image computing unit 14. In some embodiments, the HMD, AR device, or other head tracking device can provide an operator (e.g., a surgeon, a nurse or other suitable medical professional) with a display that is at least in part coupled or mounted to the head of the operator, lenses to allow a focused view of the display, and the sensing and tracking unit 16A to provide position and orientation tracking of the operator's head. The sensing and tracking unit 16A can include for example accelerometers, gyroscopes, magnetometers, motion processors, infrared tracking, eye tracking, computer vision, emission and sensing of alternating magnetic fields, and any other method of tracking at least one of position and orientation, or any combination thereof. In some embodiments, the HMD or AR device can provide image data from the camera assembly 44 to the right and left eyes of the operator. In some embodiments, in order to maintain a virtual reality experience for the operator, the sensing and tracking unit 16A, can track the position and orientation of the operator's head, generate tracking and position data 34A, and then relay the tracking and position data 34A to the image computing unit 14 and/or the computing unit 18 either directly or via the image computing unit 14.

The hand controllers 17 are configured to sense a movement of the operator's hands and/or arms to manipulate the surgical robotic system 10. The hand controllers 17 can include the sensing and tracking unit 16, circuity, and/or other hardware. The sensing and tracking unit 16 can include one or more sensors or detectors that sense movements of the operator's hands. In some embodiments, the one or more sensors or detectors that sense movements of the operator's hands are disposed in a pair of hand controllers that are grasped by or engaged by hands of the operator. In some embodiments, the one or more sensors or detectors that sense movements of the operator's hands are coupled to the hands and/or arms of the operator. For example, the sensors of the sensing and tracking unit 16 can be coupled to a region of the hand and/or the arm, such as the fingers, the wrist region, the elbow region, and/or the shoulder region. If the HMD is not used, then additional sensors can also be coupled to a head and/or neck region of the operator in some embodiments. If the operator employs the HMD, then the eyes, head and/or neck sensors and associated tracking technology can be built-in or employed within the HMD device, and hence form part of the optional sensor and tracking unit 16A as described above. In some embodiments, the sensing and tracking unit 16 can be external and coupled to the hand controllers 17 via electricity components and/or mounting hardware.

In some embodiments, the sensing and tracking unit 16 can employ sensors coupled to the torso of the operator or any other body part. In some embodiments, the sensing and tracking unit 16 can employ in addition to the sensors an Inertial Momentum Unit (IMU) having for example an accelerometer, gyroscope, magnetometer, and a motion processor. The addition of a magnetometer allows for reduction in sensor drift about a vertical axis. In some embodiments, the sensing and tracking unit 16 also include sensors placed in surgical material such as gloves, surgical scrubs, or a surgical gown. The sensors can be reusable or disposable. In some embodiments, sensors can be disposed external of the operator, such as at fixed locations in a room, such as an operating room. The external sensors can generate external data 36 that can be processed by the computing unit 18 and hence employed by the surgical robotic system 10.

The sensors generate position and/or orientation data indicative of the position and/or orientation of the operator's hands and/or arms. The sensing and tracking units 16 and/or 16A can be utilized to control movement (e.g., changing a position and/or an orientation) of the camera assembly 44 and robotic arms 42 of the robotic subsystem 20. The tracking and position data 34 generated by the sensing and tracking unit 16 can be conveyed to the computing unit 18 for processing by at least one processor 22.

The computing unit 18 can determine or calculate, from the tracking and position data 34 and 34A, the position and/or orientation of the operator's hands or arms, and in some embodiments of the operator's head as well, and convey the tracking and position data 34 and 34A to the robotic subsystem 20. The tracking and position data 34, 34A can be processed by the processor 22 and can be stored for example in the storage unit 24. The tracking and position data 34A can also be used by the control unit 26, which in response can generate control signals for controlling movement of the robotic arms 42 and/or the camera assembly 44. For example, the control unit 26 can change a position and/or an orientation of at least a portion of the camera assembly 44, of at least a portion of the robotic arms 42, or both. In some embodiments, the control unit 26 can also adjust the pan and tilt of the camera assembly 44 to follow the movement of the operator's head.

The robotic subsystem 20 can include a robot support system (RSS) 46 having a motor unit 40 and a trocar 50 or trocar mount, the robotic arms 42, and the camera assembly 44. The robotic arms 42 and the camera assembly 44 can form part of a single support axis robot system, such as that disclosed and described in U.S. Pat. No. 10,285,765, or can form part of a split arm (SA) architecture robot system, such as that disclosed and described in PCT Patent Application No. PCT/US2020/039203, both of which are incorporated herein by reference in their entirety.

The robotic subsystem 20 can employ multiple different robotic arms that are deployable along different or separate axes. In some embodiments, the camera assembly 44, which can employ multiple different camera elements, can also be deployed along a common separate axis. Thus, the surgical robotic system 10 can employ multiple different components, such as a pair of separate robotic arms and the camera assembly 44, which are deployable along different axes. In some embodiments, the robotic arms 42 and the camera assembly 44 are separately manipulatable, maneuverable, and movable. The robotic subsystem 20, which includes the robotic arms 42 and the camera assembly 44, is disposable along separate manipulatable axes, and is referred to herein as an SA architecture. The SA architecture is designed to simplify and increase efficiency of the insertion of robotic surgical instruments through a single trocar at a single insertion point or site, while concomitantly assisting with deployment of the surgical instruments into a surgical ready state, as well as the subsequent removal of the surgical instruments through a trocar 50 as further described below.

The RSS 46 can include the motor unit 40 and the trocar 50 or a trocar mount. The RSS 46 can further include a support member that supports the motor unit 40 coupled to a distal end thereof. The motor unit 40 in turn can be coupled to the camera assembly 44 and to each of the robotic arms 42. The support member can be configured and controlled to move linearly, or in any other selected direction or orientation, one or more components of the robotic subsystem 20. In some embodiments, the RSS 46 can be free standing. In some embodiments, the RSS 46 can include the motor unit 40 that is coupled to the robotic subsystem 20 at one end and to an adjustable support member or element at an opposed end.

The motor unit 40 can receive the control signals generated by the control unit 26. The motor unit 40 can include gears, one or more motors, drivetrains, electronics, and the like, for powering and driving the robotic arms 42 and the cameras assembly 44 separately or together. The motor unit 40 can also provide mechanical power, electrical power, mechanical communication, and electrical communication to the robotic arms 42, the camera assembly 44, and/or other components of the RSS 46 and robotic subsystem 20. The motor unit 40 can be controlled by the computing unit 18. The motor unit 40 can thus generate signals for controlling one or more motors that in turn can control and drive the robotic arms 42, including for example the position and orientation of each articulating joint of each robotic arm, as well as the camera assembly 44. The motor unit 40 can further provide for a translational or linear degree of freedom that is first utilized to insert and remove each component of the robotic subsystem 20 through a trocar 50. The motor unit 40 can also be employed to adjust the inserted depth of each robotic arm 42 when inserted into the patient 100 through the trocar 50.

The trocar 50 is a medical device that can be made up of an awl (which may be a metal or plastic sharpened or non-bladed tip), a cannula (essentially a hollow tube), and a seal in some embodiments. The trocar can be used to place at least a portion of the robotic subsystem 20 in an interior cavity of a subject (e.g., a patient) and can withdraw gas and/or fluid from a body cavity. The robotic subsystem 20 can be inserted through the trocar to access and perform an operation in vivo in a body cavity of a patient. The robotic subsystem 20 can be supported by the trocar 50 or a trocar mount with multiple degrees of freedom such that the robotic arms 42 and the camera assembly 44 can be maneuvered within the patient into a single position or multiple different positions.

In some embodiments, the RSS 46 can further include an optional controller for processing input data from one or more of the system components (e.g., the display 12, the sensing and tracking unit 16, the robotic arms 42, the camera assembly 44, and the like), and for generating control signals in response thereto. The motor unit 40 can also include a storage element for storing data.

The robotic arms 42 can be controlled to follow the scaled-down movement or motion of the operator's arms and/or hands as sensed by the associated sensors. The robotic arms 42 include a first robotic arm including a first end effector at distal end of the first robotic arm, and a second robotic arm including a second end effector disposed at a distal end of the second robotic arm. In some embodiments, the robotic arms 42 can have portions or regions that can be associated with movements associated with the shoulder, elbow, and wrist joints as well as the fingers of the operator. For example, the robotic elbow joint can follow the position and orientation of the human elbow, and the robotic wrist joint can follow the position and orientation of the human wrist. The robotic arms 42 can also have associated therewith end regions that can terminate in end-effectors that follow the movement of one or more fingers of the operator in some embodiments, such as for example the index finger as the user pinches together the index finger and thumb. In some embodiments, while the robotic arms of the robotic arms 42 may follow movement of the arms of the operator in some modes of control, the robotic shoulders are fixed in position in such modes of control. In some embodiments, the position and orientation of the torso of the operator are subtracted from the position and orientation of the operator's arms and/or hands. This subtraction allows the operator to move his or her torso without the robotic arms moving. Further disclosure control of movement of individual arms of a robotic arm assembly is provided in International Patent Application Publications WO 2022/094000 A1 and WO 2021/231402 A1, each of which is incorporated by reference herein in its entirety.

The camera assembly 44 is configured to provide the operator with image data 48, such as for example a live video feed of an operation or surgical site, as well as enable the operator to actuate and control the cameras forming part of the camera assembly 44. In some embodiments, the camera assembly 44 can include one or more cameras (e.g., a pair of cameras), the optical axes of which are axially spaced apart by a selected distance, known as the inter-camera distance, to provide a stereoscopic view or image of the surgical site. In some embodiments, the operator can control the movement of the cameras via movement of the hands via sensors coupled to the hands of the operator or via hand controllers grasped or held by hands of the operator, thus enabling the operator to obtain a desired view of an operation site in an intuitive and natural manner. In some embodiments, the operator can additionally control the movement of the camera via movement of the operator's head. The camera assembly 44 is movable in multiple directions, including for example in yaw, pitch and roll directions relative to a direction of view. In some embodiments, the components of the stereoscopic cameras can be configured to provide a user experience that feels natural and comfortable. In some embodiments, the interaxial distance between the cameras can be modified to adjust the depth of the operation site perceived by the operator.

The image or video data 48 generated by the camera assembly 44 can be displayed on the display unit 12. In embodiments in which the display unit 12 includes a HMD, the display can include the built-in sensing and tracking unit 16A that obtains raw orientation data for the yaw, pitch and roll directions of the HMD as well as positional data in Cartesian space (x, y, z) of the HMD. In some embodiments, positional and orientation data regarding an operator's head may be provided via a separate head-tracking unit. In some embodiments, the sensing and tracking unit 16A may be used to provide supplementary position and orientation tracking data of the display in lieu of or in addition to the built-in tracking system of the HMD. In some embodiments, no head tracking of the operator is used or employed.

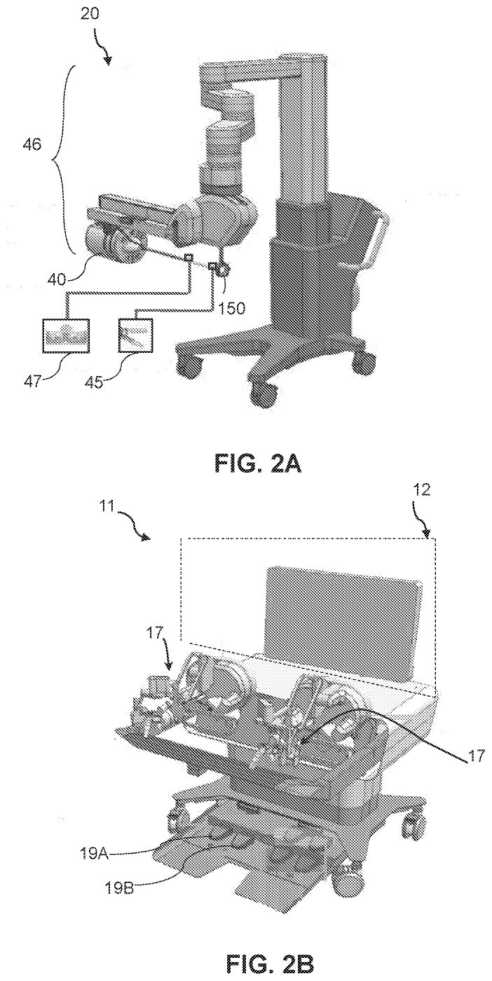

FIG. 2A depicts an example robotic assembly 20 of a surgical robotic system 10 incorporated into or mounted onto a mobile patient cart in accordance with some embodiments. In some embodiments, the robotic assembly 20 includes the RSS 46, which, in turn includes the motor unit 40, the robotic arm assembly 42 having end-effectors 45, the camera assembly 44 having one or more cameras 47, and may also include the trocar 50 or a trocar mount.

FIG. 2B depicts an example of an operator console 11 of the surgical robotic system 10 of the present disclosure in accordance with some embodiments. The operator console 11 includes a display unit 12, hand controllers 17, and may also include one or more additional controllers (e.g., foot pedals or switches) for control of the robotic arms 42, for control of the camera assembly 44, and for control of other aspects of the system.

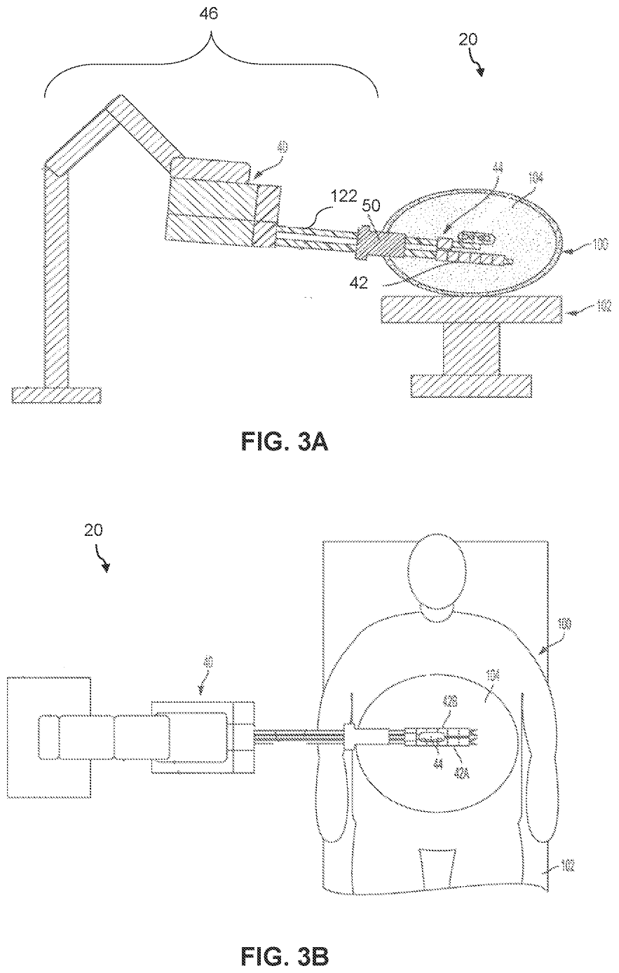

FIG. 3A schematically depicts a side view of the surgical robotic system 10 performing a surgery within an internal cavity 104 of a subject 100 in accordance with some embodiments and for some surgical procedures. FIG. 3B schematically depicts a top view of the surgical robotic system 10 performing the surgery within the internal cavity 104 of the subject 100. The subject 100 (e.g., a patient) is placed on an operation table 102 (e.g., a surgical table 102). In some embodiments, and for some surgical procedures, an incision is made in the patient 100 to gain access to the internal cavity 104. The trocar 50 is then inserted into the patient 100 at a selected location to provide access to the internal cavity 104 or operation site. The RSS 46 can then be maneuvered into position over the patient 100 and the trocar 50. In some embodiments, the RSS 46 includes a trocar mount that attaches to the trocar 50. The robotic assembly 20 can be coupled to the motor unit 40 and at least a portion of the robotic assembly can be inserted into the trocar 50 and hence into the internal cavity 104 of the patient 100. For example, the camera assembly 44 and the robotic arm assembly 42 can be inserted individually and sequentially into the patient 100 through the trocar 50. Although the camera assembly and the robotic arm assembly may include some portions that remain external to the subject's body in use, references to insertion of the robotic arm assembly 42 and/or the camera assembly into an internal cavity of a subject and disposing the robotic arm assembly 42 and/or the camera assembly 44 in the internal cavity of the subject are referring to the portions of the robotic arm assembly 42 and the camera assembly 44 that are intended to be in the internal cavity of the subject during use. The sequential insertion method has the advantage of supporting smaller trocars and thus smaller incisions can be made in patient 100, thus reducing the trauma experienced by the patient 100. In some embodiments, the camera assembly 44 and the robotic arm assembly 42 can be inserted in any order or in a specific order. In some embodiments, the camera assembly 44 can be followed by a first robot arm of the robotic arm assembly 42 and then followed by a second robot arm of the robotic arm assembly 42 all of which can be inserted into the trocar 50 and hence into the internal cavity 104. Once inserted into the patient 100, the RSS 46 can move the robotic arm assembly 42 and the camera assembly 44 to an operation site manually or automatically controlled by the operator console 11.

Further disclosure regarding control of movement of individual arms of a robotic arm assembly is provided in International Patent Application Publications WO 2022/094000 A1 and WO 2021/231402 A1, each of which is incorporated by reference herein in its entirety.

### Robotic Assembly Control

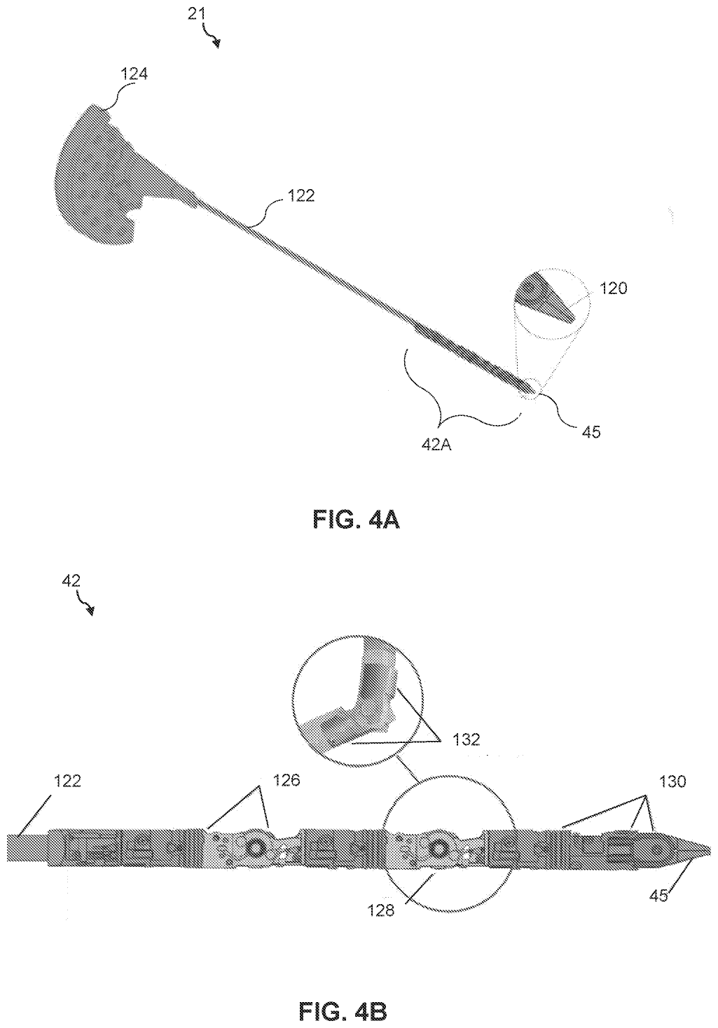

FIG. 4A is a perspective view of a robotic arm subassembly 21 in accordance with some embodiments. The robotic arm subassembly 21 includes a robotic arm 42A, the end-effector 45 having an instrument tip 120 (e.g., monopolar scissors, needle driver/holder, bipolar grasper, or any other appropriate tool), a shaft 122 supporting the robotic arm 42A. A distal end of the shaft 122 is coupled to the robotic arm 42A, and a proximal end of the shaft 122 is coupled to a housing 124 of the motor unit 40 (as shown in FIGS. 1 and 2A). At least a portion of the shaft 122 can be external to the internal cavity 104 (as shown in FIGS. 3A and 3B). At least a portion of the shaft 122 can be inserted into the internal cavity 10 (as shown in FIGS. 3A and 3B).

FIG. 4B is a side view of the robotic arm assembly 42. The robotic arm assembly 42 includes a virtual shoulder 126, a virtual elbow 128 having capacitive proximity sensors 132, a virtual wrist 130, and the end-effector 45. The virtual shoulder 126, the virtual elbow 128, the virtual wrist 130 can include a series of hinge and rotary joints to provide each arm with positionable, seven degrees of freedom, along with one additional grasping degree of freedom for the end-effector 45.

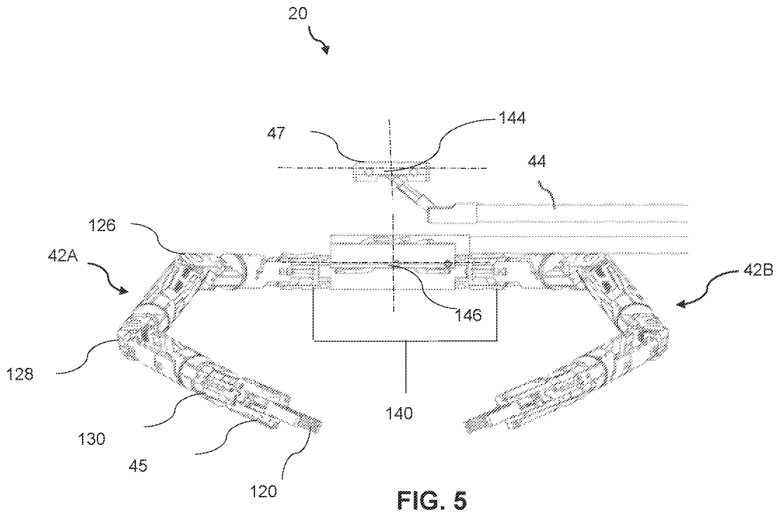

FIG. 5 illustrates a perspective front view an internal portion of the robotic assembly 20. The robotic assembly 20 includes a first robotic arm 42A and a second robotic arm 42B. The two robotic arms 42A and 42B can define a virtual chest 140 of the robotic assembly 20. The virtual chest 140 can be defined by a chest plane extending between a first pivot point 142A of a most proximal joint of the first robotic arm 42A, a second pivot point 142B of a most proximal joint of the second robotic arm 42B, and a camera imaging center point 144 of the camera(s) 47. A pivot center 146 of the virtual chest 140 lies midway along a line segment in the chest plane connecting the first pivot point 144 of the first robotic arm 42A and the second pivot point 142B of the second robotic arm. 42B.

In some embodiments, sensors in one or both of the first robotic arm 42A and the second robotic arm 42B can be used by the system to determine a change in location in three-dimensional space of at least a portion of the robotic arm. In some embodiments, sensors in one of both of the first robotic arm and second robotic arm can be used by the system to determine a location in three-dimensional space of at least a portion of one robotic arm relative to a location in three-dimensional space of at least a portion of the other robotic arm.

In some embodiments, a camera assembly 44 is configured to obtain images from which the system can determine relative locations in three-dimensional space. For example, the camera assembly may include multiple cameras, at least two of which are laterally displaced from each other relative to an imaging axis, and the system may be configured to determine a distance to features within the internal body cavity. Further disclosure regarding a surgical robotic system including camera assembly and associated system for determining a distance to features may be found in International Patent Application Publication No. WO 2021/159409, entitled “System and Method for Determining Depth Perception In Vivo in a Surgical Robotic System,” and published Aug. 12, 2021, which is incorporated by reference herein in its entirety. Information about the distance to features and information regarding optical properties of the cameras may be used by a system to determine relative locations in three-dimensional space.

### Pitch Systems

Pitch systems and methods described and depicted herein may be employed with a robotic surgical system as described above. Some pitch systems and methods described herein may be particularly beneficial with surgical robotic systems, such as that above, having robotic arms and a robotic chest with many different degrees of freedom for movement. In such systems, a total pitch orientation range of about 50 degrees would likely be sufficient in view of the many different degrees of freedom of movements and configurability of the robot arm assembly. The overall reduced size, and relatively light weight of the pitch system would enable a patient cart incorporating the pitch system to be more mobile and easy to transport (e.g., through doorways of a hospital). One of ordinary skill in the art in view of the present disclosure would appreciate that pitch systems and methods described and depicted herein are not limited to use with a surgical robotic system as described herein, but could also be employed in other surgical robotic systems, in surgical systems that are a hybrid of robotic and manual, and in nonsurgical robotic systems in some embodiments.

FIG. 6 is a side view of a robot support system (RSS) 46′ incorporated into or mounted onto a mobile patient cart in accordance with some embodiments. The RSS 46′ includes a pitch system 200 that controls pitch angle motion about a virtual center 202 and a yaw system 300 that controls yaw motion about yaw axis 302 of systems, subsystems, units, and/or components mounted to or supported by a positioning arm 240, which may include one or more motor units 40 and a trocar mount 150. When in use, one or more robotic arm assemblies and a camera assembly (not shown) may be mounted to or on the positioning arm 240. The virtual center 202 can be defined as an intersection point at an intersection of the yaw axis 302 and a pitch axis (not shown). The pitch axis is a transverse or lateral axis passing through the pitch system 200. Rotation about the pitch axis is called pitch. Pitch changes the vertical direction that the insertion axis 304 is pointing. The pitch axis can pass through the virtual center 202. The pitch axis provides a reference allowing the pitching system 200 to pitch up and down an insertion axis 304 relative to the pitch axis. The insertion axis 304 is an axis that extends parallel to the positioning arm 240 and extends through a center of the trocar mount 150. In use, one or more robotic arm assemblies and/or a camera assembly supported by the positioning arm 240 may collectively be inserted through the trocar mount 150 and a trocar 50 mounted to the trocar mount 150 and into the patient along the insertion axis 304 or an axis parallel to the insertion axis 304 that also passes through the trocar.

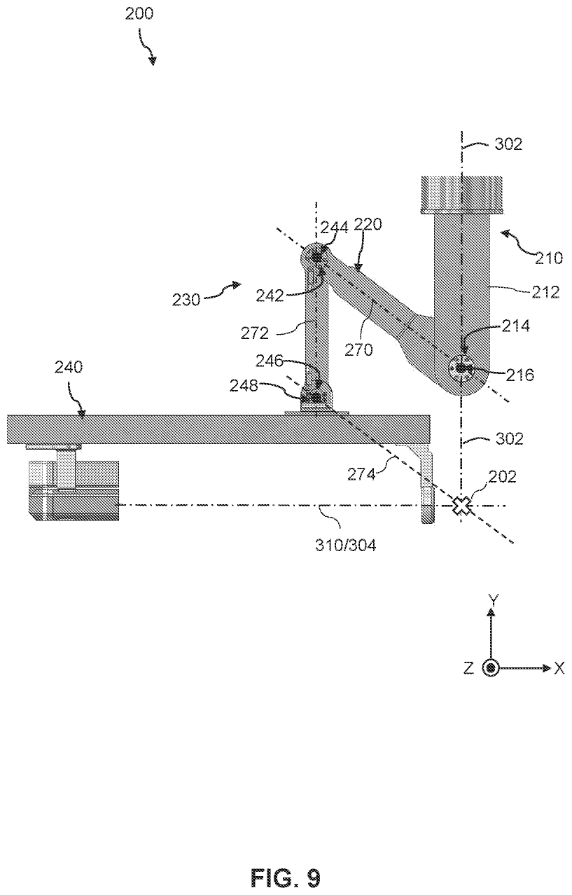

The pitch system 200 generates and controls pitch motion about the virtual center 202. This pitch motion enables systems, subsystems, units and/or other components mounted to or supported by the positioning arm 240, for example the robotic arm subassembly 21 shown of FIG. 4A to be pitched upward or downward relative to the virtual center 202, which usually corresponds to an incision site or a site within the trocar 50. Relationships among the yaw axis 302, the insertion axis 304, and the virtual center 202 are also described with respect to FIG. 9.

Although pitch system 200 is described and depicted herein as incorporated into or configured to be mounted onto a mobile patient cart, one of ordinary skill in the art in view of the present disclosure will understand that pitch systems described herein are not limited to use with such a mobile patient cart. Pitch systems described herein may be incorporated into or configured to be mounted onto a mobile patient cart having a different configuration, a different mobile support, or a different immobile support in accordance with some embodiments.

Although pitch system 200 is described and depicted herein as supporting positioning arm 240, one of ordinary skill in the art in view of the present disclosure will understand that pitch systems described herein may be used with other designs or types of positioning arms or other supports for portions of a surgical system to be inserted into a patient's body. One of ordinary skill in the art, in view of the present disclosure, will understand that pitch systems described herein are not limited to use with robotic arm assemblies and camera assemblies described herein. Pitch systems described herein may be used with other types of robotic surgical systems, other types of camera systems, and non-robotic systems in accordance with some embodiments.

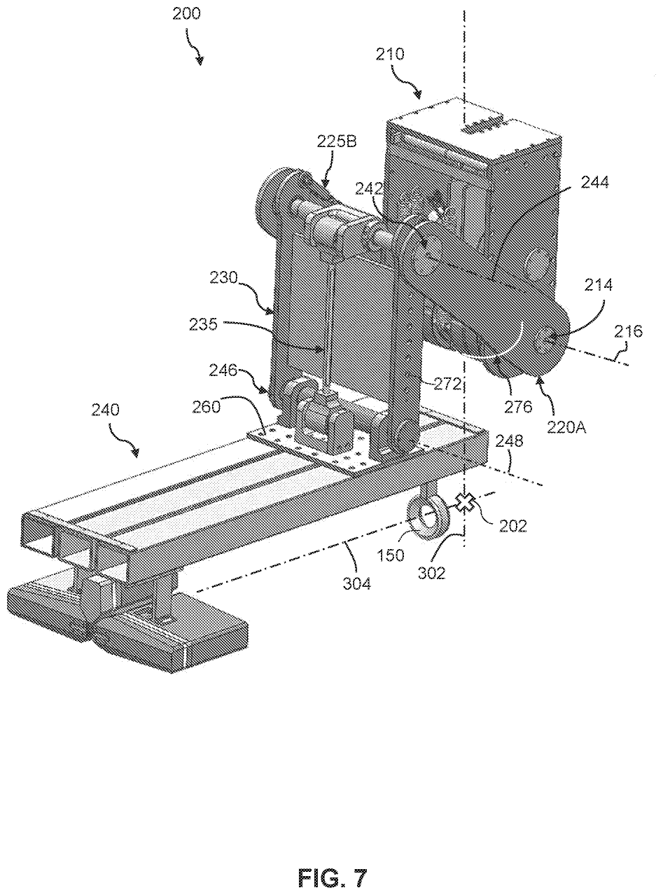

FIG. 7 is a perspective view of the pitch system 200 in accordance with some embodiments. The pitch system 200 includes a pitching housing assembly 210, at least one angled linkage body (e.g., first and second angled linkage side plates 220a, 220b), at least one angled rigid member (e.g., first and second side angled rigid members 225a, 225b, at least one parallel linkage body (e.g., first and second parallel linkage side plates 230a, 230b), at least one parallel rigid member (e.g., parallel rigid member 235), and positioning arm 240 or a mounting (e.g., a mounting plate) 260 for the positioning arm 240. As noted above, references “to a positioning arm” or “the positioning arm” should be interpreted as references to “a positioning arm or a mounting for the positioning arm” or “the positioning arm or the mounting for the positioning arm,” where appropriate, throughout this application. The pitching housing assembly 210 has a first end configured to be connected with, configured to be connected to, connected with, or connected to the yaw system 300 (shown in FIG. 6) that defines the yaw axis 302. The pitch system 200 also includes a first rotary joint 216 having a first rotation axis 216, which may be included in the pitch housing assembly 210, a second rotary joint 242 having a second rotation axis 244, and a third rotary joint 246 having a third rotation axis 248.

The first and second angled linkage side plates 220a, 220a, which are included in the at least one angled linkage body, each have a proximal end rotationally coupled to the pitch housing assembly 210 via the first rotary joint 214, and a distal end rotationally coupled to a corresponding one of the first and second parallel linkage side plates 230a, 230a, which are included in the at least parallel linkage body, via the second rotary joint 242. In the context of components extending between and connecting the pitch housing assembly 210 to the positioning arm 260, a proximal end of a component is more closely connected to the pitch housing and a distal end of the component is further away or less directly connected to the pitch housing than the proximal end. The at least one angled linkage body (e.g., first and second angled linkage side plates 220a, 220b, collectively) has a first linkage body axis 270 that is defined as a line perpendicular to the second rotation axis 244 and extending from the second rotation axis 244 at the second rotary joint 242 through the first rotation axis 216 at the first rotary joint 214 and intersecting the yaw axis 302 joint (see FIG. 9). The first linkage body axis 270 is never parallel to the yaw axis 302, but is instead at a nonzero angle relative to the yaw axis (see FIG. 9), which is why at least one linkage body that connects to a pitch housing at a first rotary joint is referred to as at least one angled linkage body herein.

The first and second parallel linkage side plates 230a, 230a, which are included in the at least one parallel linkage body, each have a proximal end rotationally coupled to corresponding respective angled linkage side plate 220a, 220a via the second rotary joint 242, and have a distal end rotationally coupled to the positioning arm 240 via the third rotary joint 246. The at least one parallel linkage body (e.g., the first and second parallel linkage side plates 230a, 230b, collectively) has a second linkage body axis 272 (see FIG. 9) that is defined as a line perpendicular to and extending from the third rotation axis 248 at the third rotary joint 246 to the second rotation axis 244 and intersecting the first linkage body axis 270. The second linkage body axis 272 is always parallel to the yaw axis 302 (see FIG. 9), which is why at least one linkage body that connects to positioning arm or a mount for a positioning arm at the third rotary joint is referred to as at least one parallel linkage body herein.

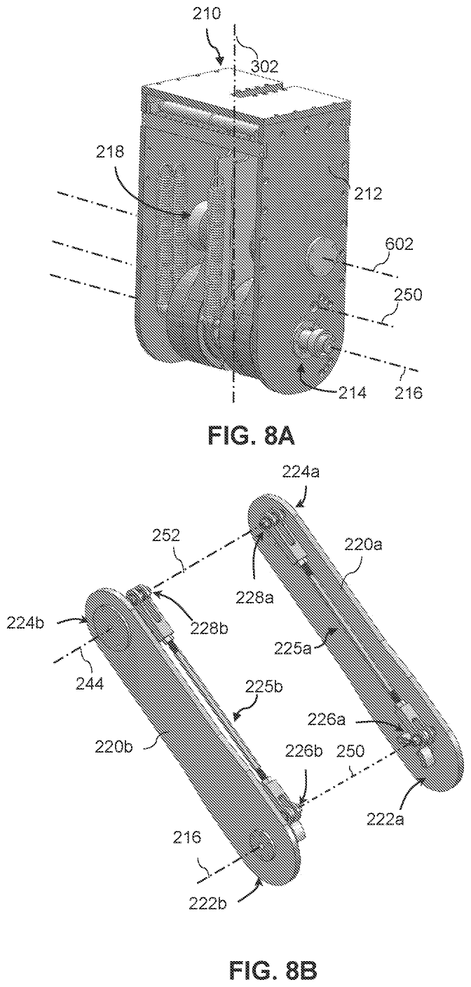

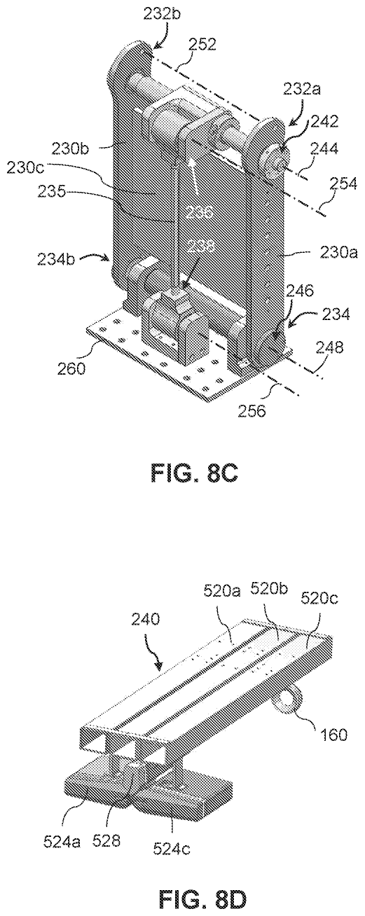

Turning to the rigid members, at least one angled rigid member (e.g., first and second side angled rigid members 225a, 225b) of the pitch system 200 is configured to cause rotation of the at least one parallel linkage body (e.g., the first and second parallel linkage side plates 230a, 230b) in response to a rotation of the at least one angled linkage body (e.g., first and second angled linkage side plates 220a, 220b). The at least one parallel rigid member (e.g., central parallel rigid member 235) is configured to cause a rotation of the positioning arm 240 in response to a rotation of the at least one parallel linkage body (e.g., the first and second parallel linkage side plates 230a, 230b). The at least one angled rigid member (e.g., first and second side angled rigid members 225a, 225b) and the at least one parallel rigid member (e.g., parallel rigid member 235) are also configured to constrain motion of the at least one angled linkage body (e.g., first and second angled linkage side plates 220a, 220b), the at least one parallel linkage body (e.g., the first and second parallel linkage side plates 230a, 230b), and the positioning arm 240 relative to each other and relative to the pitch housing 212 to maintain an orientation of the second linkage body axis 272 parallel to the yaw axis 302 and to maintain an orientation of the first linkage body axis 270 parallel to a line 274 perpendicular to the third rotational axis 248 extending from the virtual center 202 to the third rotational axis 248, also referred to herein as the third axis-virtual center line, during rotation of the at least one angled linkage body (e.g., first and second angled linkage side plates 220a, 220b) relative to the pitch housing 212. Further explanation of how the rigid members constrain motion is provided below with respect to FIG. 9. Further explanation of the rigid members is provided below with respect to FIGS. 8B, 8C, 17A-21, 24A and 24B.

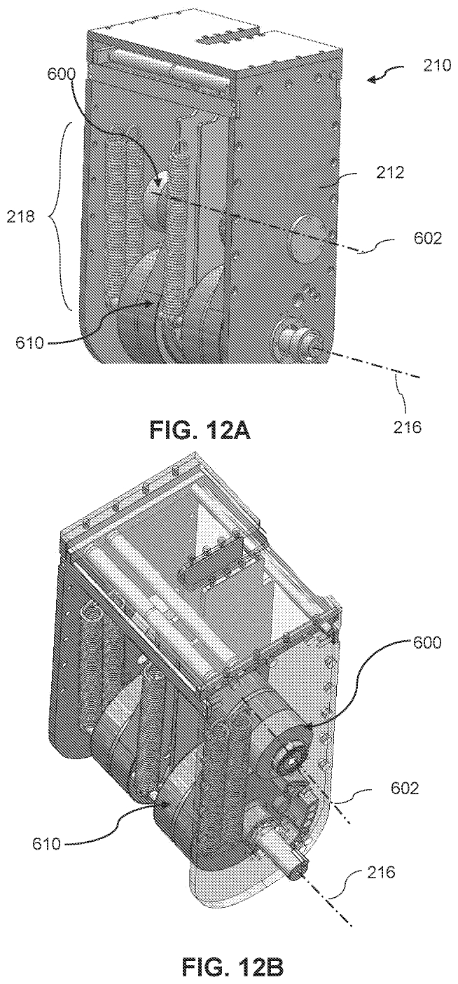

FIG. 8A depicts a pitch housing assembly 210 in accordance with some embodiments. As shown in FIG. 8A, the pitching housing assembly 210 includes a pitch housing 212 that supports various components within the pitch housing 212, the first rotary joint 214 having the first rotational axis 216 perpendicular to and intersecting the yaw axis 312, and an actuator assembly 218 configured to drive a rotation of the at least one angled linkage body (e.g., first and second angled linkage side plates 220a, 220b) relative to the pitch housing 212 about the first rotary joint 214 to cause an angled linkage body rotation. In some embodiments, the actuator assembly 218 drives rotation about a drive axis 602, which may also be referred to herein as an output axis or a motor axis, and the rotation about the drive axis 602 is coupled to and drives rotation about the first rotary axis 216 as described in further detail below with respect to FIGS. 12A-14B.

FIG. 8B depicts first and second angled linkage side plates 220a, 220b, which are included in the at least one angled linkage body in some embodiments. The first and second angled linkage side plates 220a and 220b each have a proximal end 222a and 222b rotationally coupled to the pitch housing 212 at the first rotary joint 214 (e.g., as shown in FIG. 8A), and each have a distal end 224a and 224b rotationally coupled to the central parallel linkage body 230 at the second rotary joint 242 (e.g., as shown in FIG. 8C).

FIG. 8B also depicts a first side angled rigid member 225a and a second side angled rigid member 225b, which are included in the at least one angled rigid member in some embodiments. As mentioned above, the first side and second side angled rigid members 225b and 225b can cause a parallel linkage body rotation of the at least one parallel linkage body (e.g., the first and second parallel linkage side plates 230a, 230b) relative to the angled linkage side plates 220a and 220b about the second rotary joint 242 due to the angled linkage body rotation of the first and second angled linkage side plates 220a, 220b. The first side and second side angled rigid members 225a and 225b each have a proximal end, 226a and 226b respectively, rotationally coupled to the pitch housing 212 at a first pivot axis 250 (see FIG. 8A) offset from the first rotation axis 216. Each of the first and second side angled rigid members 225a and 225b also has a distal end, 228a and 228b respectively, rotationally coupled to a proximal end 232a and 232b of a corresponding first or second parallel linkage side plate 230a and 230b at a second pivot axis 252 parallel to and offset from the second rotation axis 244, as further described with respect to FIG. 8C. Despite being depicted together in FIG. 8B, the first and second angled linkage side plates 220a, 220b are not directly connected to the first and second side angled rigid members 225a and 225b.

As shown in FIG. 8C, in some embodiments at least one parallel linkage body includes the first parallel linkage side plate 230a and the second parallel linkage side plate 230b. As also shown in FIG. 8C, an additional plate 230c may connect the first and second parallel linkage side plates 230a, 230b, for example, to provide any of strength, rigidity, and support an/or to reduce torsion. Each of the first and second parallel linkage side plates 230a and 230b has a proximal end 232a and 232b rotationally coupled to the respective corresponding first or second angled linkage side plate 220a and 220b at the second rotary joint 242, and has a distal ends 234a and 234b rotationally coupled to the positioning arm 240 directly or via a mounting (e.g., a mounting plate) 260 at a third rotary joint 246. The third rotary joint 246 has a third rotation axis 248 parallel to the first rotation axis 216.

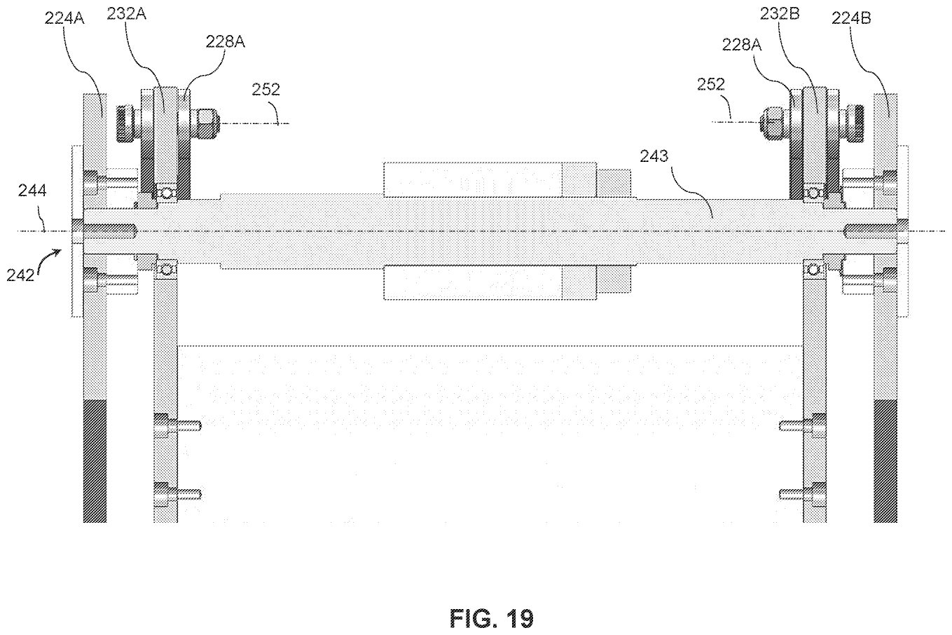

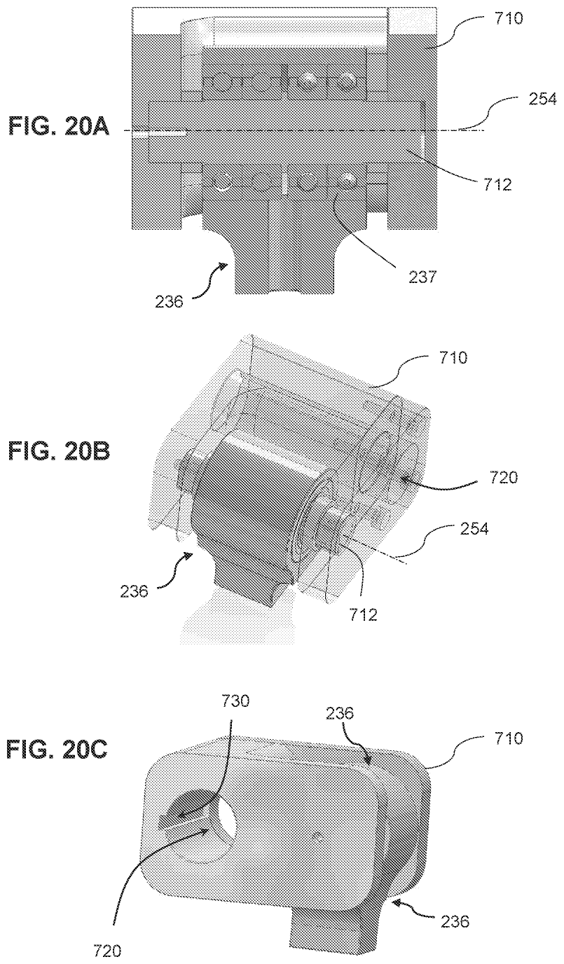

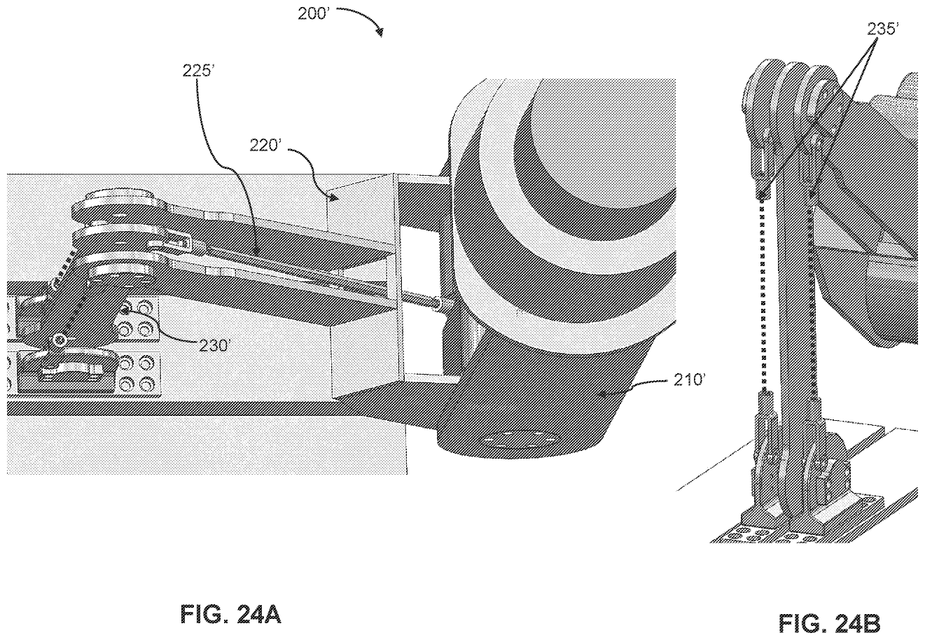

In some embodiments, the at least one parallel rigid member is a central rigid member 235 as shown in FIG. 8C. As described above, the central parallel rigid member 235 can cause a rotation of the positioning arm 240 relative to the parallel linkage side plates 230A and 230B due to a rotation of the first and second parallel linkage side plates 230a, 230b. The central parallel rigid member 235 has a proximal end 236 rotationally coupled with the angled linkage side plates 220a and 220b at a third pivot axis 254 parallel to and offset from the second rotation axis 244. In some embodiments, a proximal end of at least one parallel rigid member is directly rotationally connected to the at least one angled linkage body (see FIGS. 24A and 24B below). In other embodiments, a proximal end of at least one parallel rigid member is rotationally connected to the at least one angled linkage body indirectly through one or more elements or components rotationally locked to the at least one angled linkage body (see, e.g., description of FIGS. 19-20C below). The distal end 238 of the central parallel rigid member 235 is rotationally coupled to the positioning arm 240 or to a mounting (e.g., mounting plate) 260 configured to be attached to the positioning arm 240 at a fourth pivot axis 256 offset from and parallel to the third rotation axis 248.

FIG. 8D depicts a positioning arm 240 including insertion rails 520a, 520b, 520c, instrument drives and cassettes 524a, 524c, each for a different robotic arm, and a camera drive and cassette 528 for a camera assembly in accordance with some embodiments. The instrument drives and cassettes 524a, 524c, and the camera drive and cassette 528 are advanced, individually or as a group, along respective insertion rails 520a, 520b, 520c, during use. As shown, a trocar mount 150 may be attached to or included on the positioning arm 240.

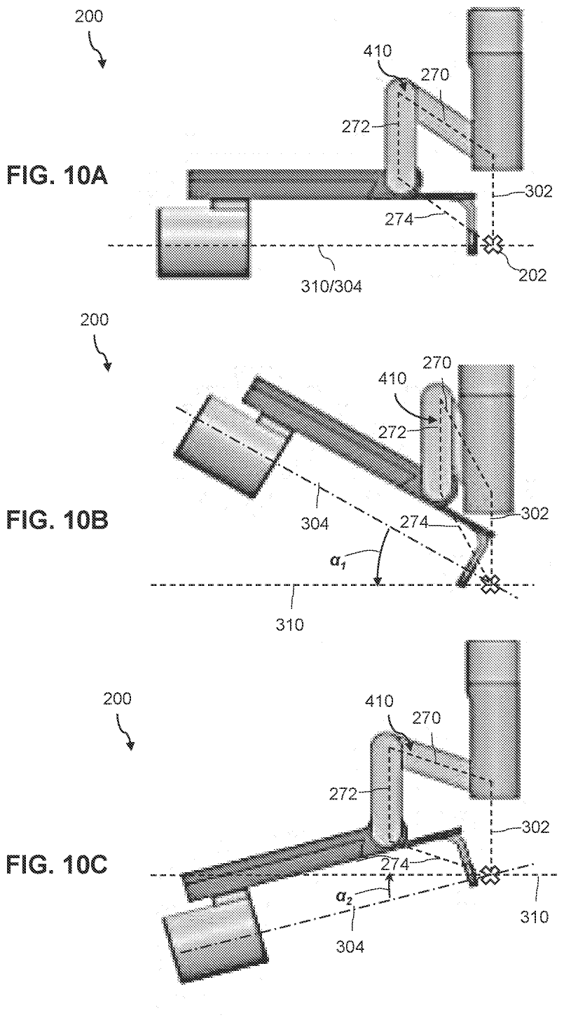

FIG. 9 is a side view of the pitch system 200 in accordance with some embodiments. When in use, the pitch housing assembly 210, the at least one angled rigid member (e.g., first and second side angled rigid members 225a, 225b), and the at least one parallel rigid member (e.g., central parallel rigid member 235) are configured to constrain motion of the at least one parallel linkage body (e.g., the first and second parallel linkage side plates 230a, 230b, collectively), and the positioning arm 240 or the mount 260 for the positioning arm to maintain an orientation of the second linkage body axis 272 parallel to the yaw axis 302 and to maintain an orientation of the first linkage body axis 270 parallel to the line 274 perpendicular to the third rotational axis 248 extending from the virtual center 202 to the third rotational axis 248 during rotation of the at least one angled linkage body (e.g., first and second angled linkage side plates 220a, 220b, collectively) relative to the pitch housing 212. For example, when in use, first and second angled linkage side plates 220a, 220b can be rotated relative to the pitch housing 212 about the first rotation axis 216. Due to this rotation of the first and second angled linkage side plates 220a, 220b with respect to the pitch housing 212 and their rotational coupling to the first and second parallel linkage side plates 230a, 230b, this also causes the first and second side angled rigid members 225a, 225b, which are rotationally connected to the pitch housing 212 and the first and second parallel linkage side plates 230a, 230b, to rotate relative to the pitch housing 212 at the first pivot axis 250 and to rotate relative to the first and second parallel linkage side plates 230a, 230b at the second pivot axis 252. Assuming a particular yaw orientation, the at least one parallel linkage body (e.g., the first and second parallel linkage side plates 230a, 230b) can be translated in the X-Y plane of FIG. 9 while remaining parallel to the yaw axis 302. Movements of the at least one angled linkage body e.g., first and second angled linkage side plates 220a, 220b, collectively), the at least one parallel linkage body 230 (e.g., the first and second parallel linkage side plates 230a, 230b, collectively), are constrained to form a parallelogram shape created by the first linkage body axis 720, the second linkage body axis 272, a line 274 perpendicular to the third rotational axis 248 and extending from the virtual center 202 to the third rotational axis 248, and the yaw axis 302. As illustrated in FIG. 9, a horizontal line 310 is parallel to the insertion axis 304 at 0 degree pitch. Example movements of the pitch system 200 to pitch up and down the insertion axis 304 relative the horizontal line 310 constrained by a respective parallelogram are further described with respect to FIGS. 10A-10C.

FIG. 10A depicts the pitch system 200 adjusted with the insertion axis 304 perpendicular to the yaw axis 302 and parallel to the horizontal line 310 passing through the virtual center 202 in accordance with some embodiments. For illustrative purposes and ease of description, in this example, the yaw axis 302 is assumed to be vertical. FIG. 10B depicts the pitch system 200 adjusted to pitch down the insertion axis 304 to form a first angle α₁ relative to the horizontal line 310. As depicted, the pitch angle α₁ is about −30°. FIG. 10C depicts the pitch system 200 adjusted to pitch up the insertion axis 304 to form a second angle α₂ to the horizontal line 310. As depicted, the pitch angle α₁ is about +15°. Notably, the first and second angles are not limited to the examples provided herein. For illustrative purposes, portions of the first linkage body axis 270, the second linkage body axis 272, the line 274 extending from the third rotational axis to the virtual center 202, and the yaw axis 302 that form a parallelogram shape are illustrated in FIG. 10A. As shown in FIGS. 10A-10C, the first linkage body axis 270, the second linkage body axis 272, the line 274, and the yaw axis 302 form a parallelogram 410 that maintains a constant position of the virtual center 202 for different orientations of the insertion axis 304, but whose shape changes depending on an orientation of the first linkage body axis 270 relative to the yaw axis 302.

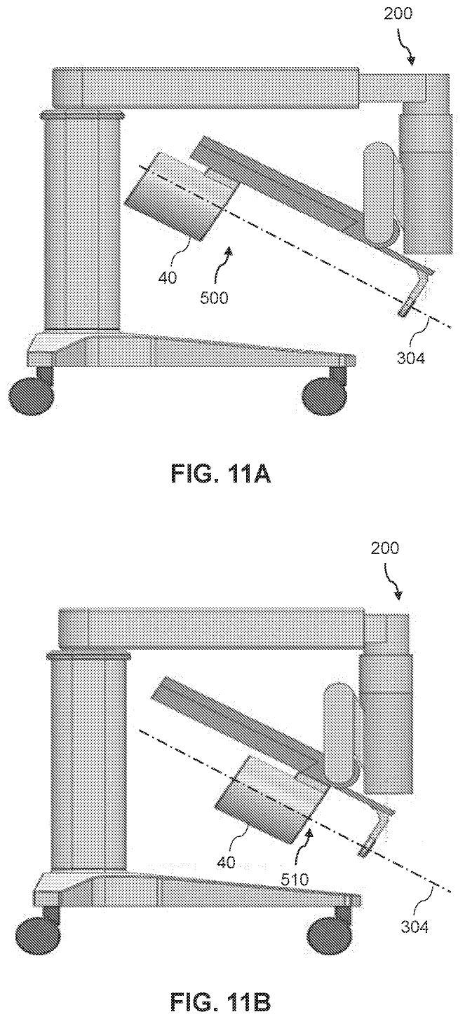

FIG. 11A depicts a motor unit 40 at a retraction position 500 away from an internal cavity of a subject in accordance with some embodiments. FIG. 11B schematically depicts the motor unit 40 at an insertion position 510 for insertion on or more robotic arm subassemblies or camera assemblies into the internal cavity in accordance with some embodiments. The robotic arm subassemblies and camera assembly are omitted for clarity.

FIG. 12A depicts an example pitch housing assembly 210 including an actuator assembly 218 in accordance with some embodiments. FIG. 12B depicts the pitch housing assembly 210 with one pitch housing side plate shown as translucent and a pitch housing top plate shown as transparent for illustrative purposes. The actuator assembly 218 includes at least one motor subassembly 600 configured to drive an output rotation about the output axis 602, also known as motor axis or drive axis, relative to the pitch housing 212 in accordance with some embodiment. In some embodiments, the rotation about the output axis 602 is coupled to a rotation about the first rotation axis 216. In some embodiments, at least one tape drive 610 couples rotation about the first rotation axis 216 with rotation about the output axis 602.

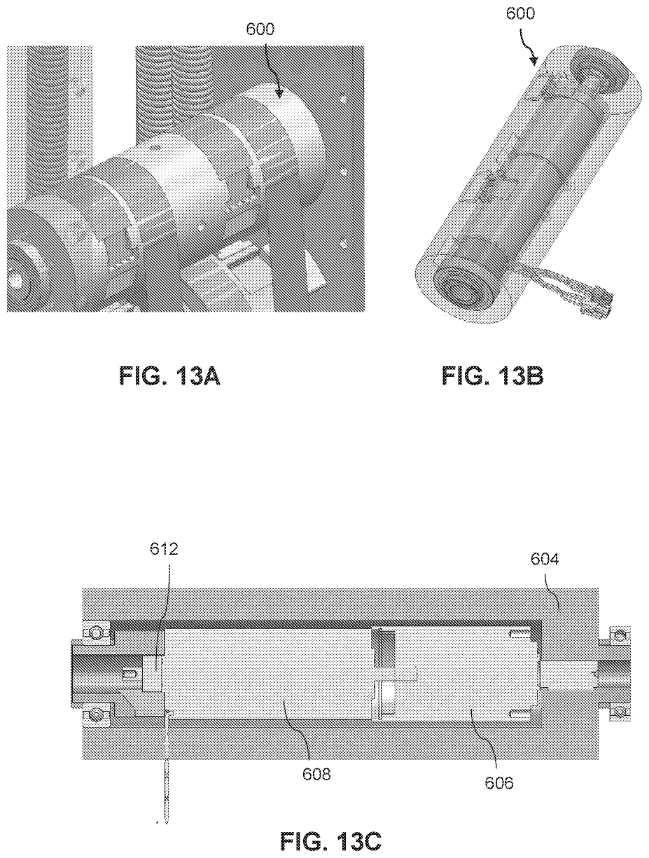

FIG. 13A depicts a motor subassembly 600 within the pitch housing assembly 210 in accordance with some embodiments. As shown in FIGS. 13B and 13C, the motor subassembly 600 includes a motor pulley 604, a gearhead 606, a motor 608, and an encoder 612 in accordance with some embodiments. The gearhead 606, the motor 608, and the encoder 612 are all disposed within the motor pulley 604.

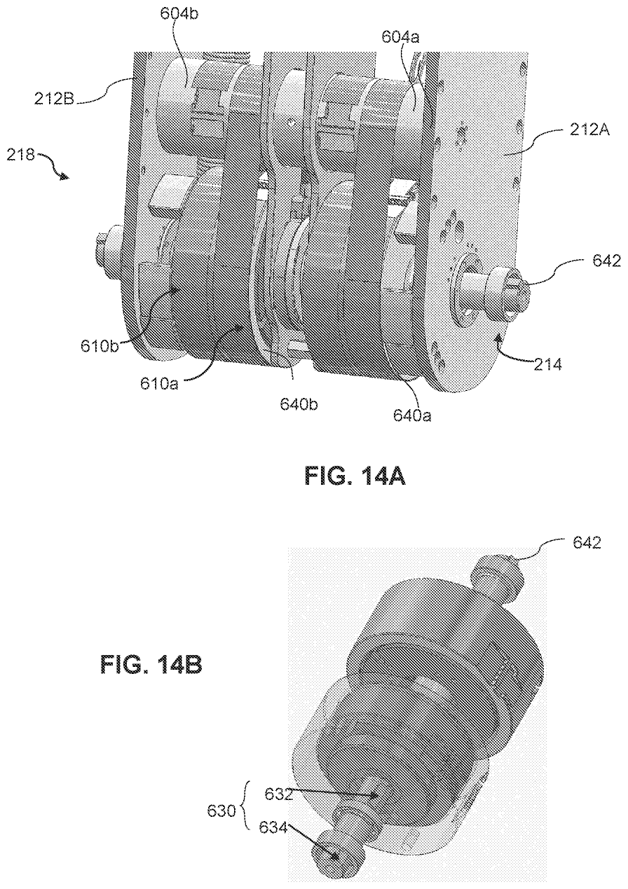

In some embodiments, the actuator assembly 218 includes two motor pulleys 604a, 604b as shown in FIG. 14A. Each motor pulley 604a, 604b is coupled to a corresponding output pulley 640a, 640b via at least one tape drive. As depicted, each motor pulley 604a, 604b is coupled to a corresponding output pulley 640a, 640b via two drive tapes (e.g., 610a, 610b) each affixed to the motor pulley 604a, 604b, at one and an affixed to the corresponding output pulley 640a, 640b at the opposite end. In some embodiments, a radius of a motor pulley may be different from a radius of a corresponding output pulley resulting in a speed reduction. For example, the radius of the motor pulley and the output pulley may be selected for about a 2:1 speed reduction in some embodiments. FIG. 14B depicts the output pulley 640a, 640b in accordance with some embodiments.

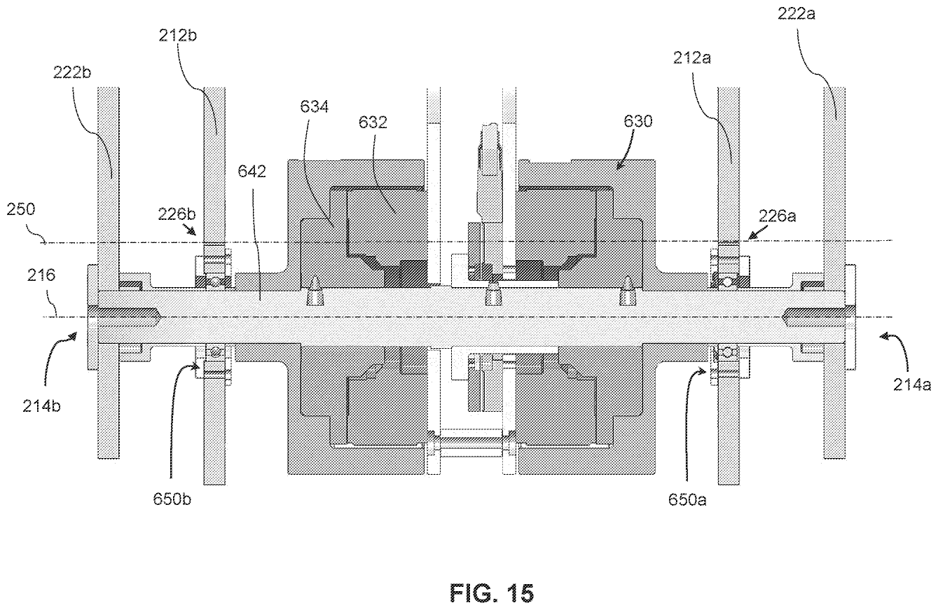

The actuator assembly 218 includes output pulleys 640a, 640b rotationally locked to a rotary shaft 642 of the first rotary joint 214 (see FIG. 15). The rotary shaft 642 of the first rotary joint 214 is rotationally locked to the proximal end of the at least one angled linkage body (e.g., to the proximal end 222a, 222b of each of the first and second angled linkage side plates 220a, 220b shown in FIG. 8B and FIG. 15). The actuator assembly 218 can includes a braking system 630 configured for braking of the rotary shaft 642 of the first rotary joint 214 relative to the pitch housing 212. The braking system 630 includes a brake rotor 634 fixed to the rotary shaft 642 of the first rotary joint 214 and a brake stator 632. The pitch housing 212 includes a first pitch housing side plate 213a and a second pitch housing side plate 213b. The rotary shaft 642 is not physically locked to the first and second pitch housing side plates 213a, 213b, but instead is separated from the first and second pitch housing side plates 213a, 213b by bearings 650a, 650b. The rotary shaft 642 is rotationally locked to proximal ends 222a, 222b of the first and second angled linkage side plates.

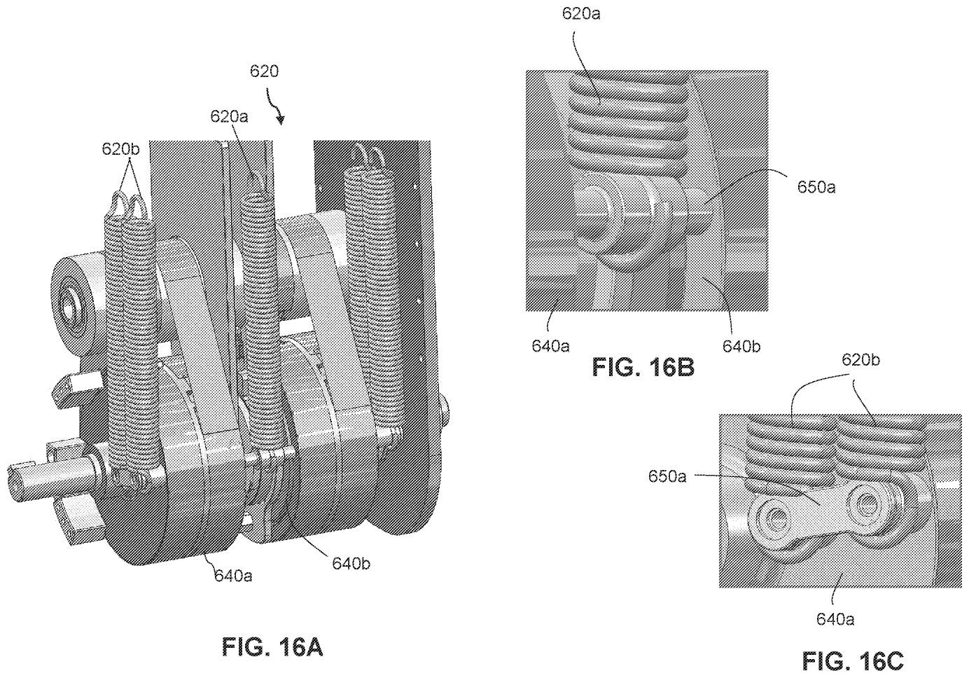

In some embodiments, extension springs may be employed in the pitch housing assembly to offset a torsional moment due to weights of downstream components. FIG. 16A depicts a central spring 620a and side springs 620b used in the pitch housing assembly 210 in accordance with some embodiments. FIG. 14B shows a middle spring 620a connected to output pulleys 640a, 640b via an attachment post 650a in accordance with some embodiments. FIG. 13C depicts side springs 620b connected to output pulley 640a via attachment posts 650b in accordance with some embodiments. Torsional moment created by the weight of the downstream components (e.g., remaining components of the pitch system 200 and other components supported by the pitch system) is offset by the extension springs 620a, 620b. The springs 620a, 620b are connected to the output pulleys 640a, 640b at specified distances from the first rotation axis via the attachment posts 650a, 650b. In some embodiments, these extension springs 620 can be used to achieve or help achieve required brake and drive tape safety factors.

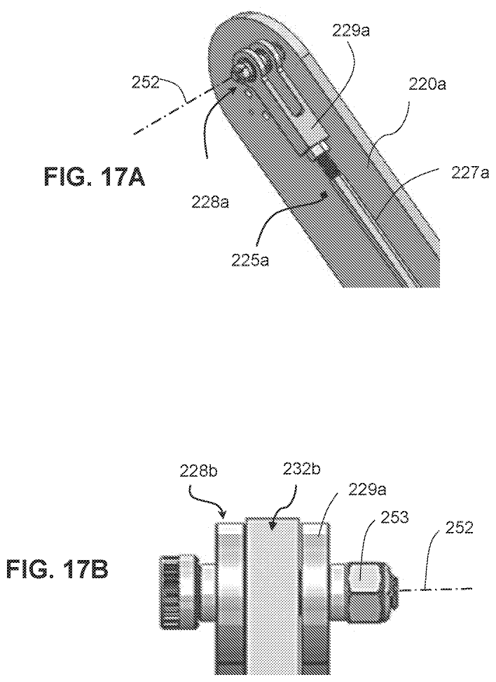

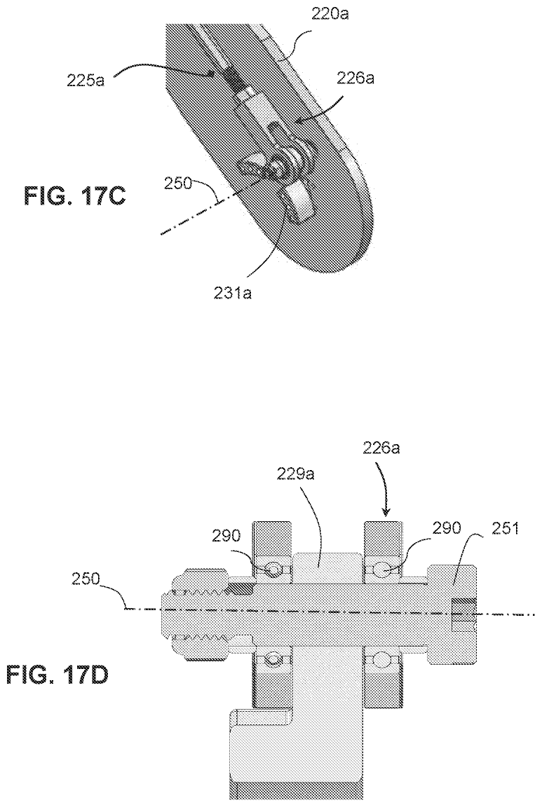

FIG. 17A is a detail of FIG. 8B including a distal portion of first side angled rigid member 225a and first angled linkage side plate 220a. In some embodiments, each of the first and second side angled rigid members 225a and 225b is in the form of a turnbuckle with an elongated center portion 227a that is threadedly attached at each end to an end portion 229a. A tension or a length of the angled rigid member would be adjustable via rotation of the center portion 227a of the turnbuckle relative to the end portions 229a.

FIG. 17B depict the distal end 228a of an angled rigid member (e.g., first side angled rigid member 225b) rotationally coupled to a proximal end (e.g., 232b) of a parallel linkage body (e.g., first parallel linkage side plate 230b) at a second pivot axis 252 in accordance with some embodiments. In some embodiments, bearings (not shown) separate the angled rigid member 225b from an axle shaft 253 at the second pivot axis 252. In some embodiments, the axle shaft 253 is formed, in part, by a locking bolt. FIG. 17C is a detail of FIG. 8B including a proximal end 226a of first side angled rigid member 225a and first angled linkage side plate 220a. FIG. 17D depicts the proximal end 226a of the first side angled rigid member 225 coupled to an offset plate 231a configured to attach to the pitch housing. In some embodiments, bearings 290 separate the proximal end 226a of the angled rigid member 225b from an axle shaft 251 having the first pivot axis 250. In some embodiments, the axle shaft 251 is part of a locking bolt.

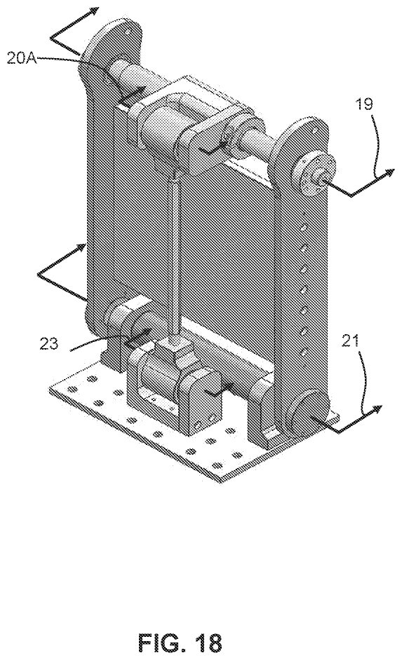

FIG. 18 depicts a portion of the pitch system 200 to and identifies cross-sectional views depicted in of FIGS. 19, 20A, 21 and 23.

FIG. 19 is a cross-sectional view of the angled linkage side plates 220A and 220B, the angled rigid members 225a and 225b, the parallel linkage side plates 230a and 230b, and the second rotary joint 242 in accordance with some embodiments. The distal ends 224a and 224b of the angled linkage side plates 220a and 220b are rotationally coupled to the proximal ends 232a and 232b of parallel linkage side plates 230a and 230a by the second rotary joint 242, which includes the second rotary joint shaft 243. The distal ends 228a and 228b of the angled rigid members 225a and 225b are rotationally coupled to the proximal ends 232a and 232b of the parallel linkage side plates 230a and 230b at the second pivot axis 252 parallel to and offset from the second rotation axis 244. The second rotary joint shaft 243 is rotationally locked to the angled linkage side plates 225A and 225B as further described with respect to FIGS. 20A-20C.

FIGS. 20A-20C depict the proximal end 236 of the central parallel rigid member 235, and a first mounting bracket 710 including a first axle shaft 712 in accordance with some embodiments. The proximal end 236 of the central parallel rigid member 235 is rotationally coupled to the first mounting bracket 710 at the third pivot axis 254 via the first axle shaft 712 in accordance with some embodiments. In some embodiments, the proximal end 236 of the central parallel rigid member 235 includes first bearings 237 for contacting the first axle shaft 712 of the first mounting bracket 710.

The first mounting bracket 710 is rotationally locked to the second rotary joint shaft 243 (not shown). In some embodiments, the second rotary joint shaft 243 (shown in FIG. 19) extends through a channel 720 of the first mounting bracket 710 and the first mounting bracket 710 is rotationally locked to the second rotary joint shaft 243 by interaction between a key on the second rotary joint shaft 243 (not shown) and a corresponding keyway 730 on the first mounting bracket 710. The second rotary shaft 243 is itself rotationally locked to the angled linkage side plates 220a and 220b, meaning that the proximal end 236 of the parallel rigid member 235 is rotationally coupled, via the first mounting bracket 710 and the second rotary joint shaft 243, to the parallel linkage side plates 230a, 230b at the third pivot axis 254.

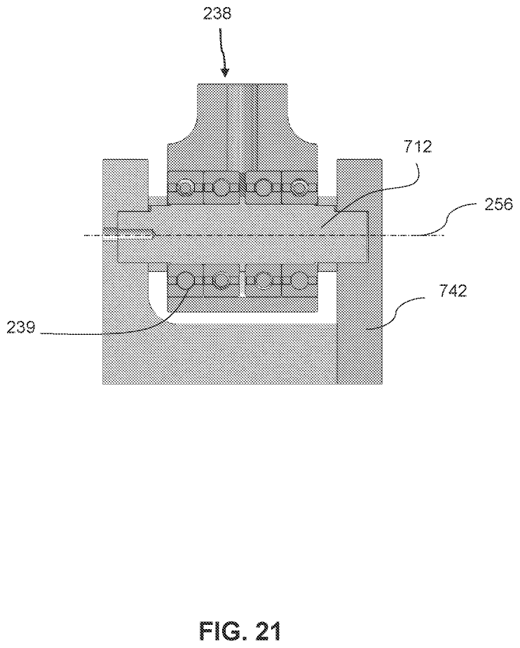

FIG. 21 is a cross-sectional view of the distal end 238 of the parallel rigid member 235 and a second mounting bracket 740 including a second shaft 742 in accordance with some embodiments. The distal end 238 of the parallel rigid member 235 is rotatably coupled with the second mounting bracket 740 at the fourth pivot axis 256 via the second shaft 742. In some embodiments, the distal end 238 of the parallel rigid member 325 includes second bearings 239 for contacting the second shaft 742. The second mounting bracket 740 is affixed to or connected to the mounting 260 for the positioning arm 240 (e.g., shown in FIG. 8C) or to the positioning arm 240, meaning that it rotatably couples the distal end 238 of the central rigid member 235 with the mount 260 for the positioning arm or with the positioning arm 240 at the fourth pivot axis 256 in accordance with some embodiments.

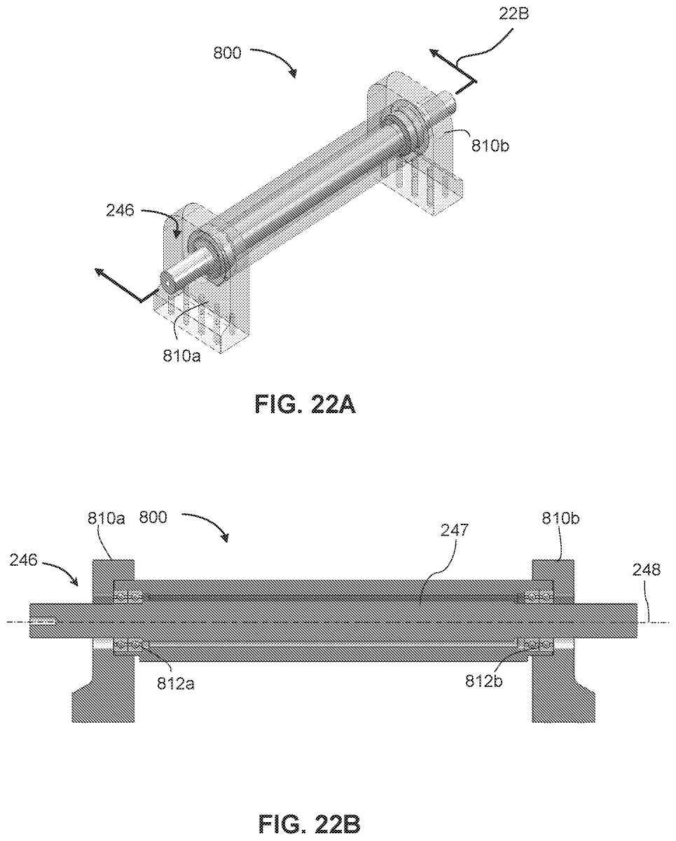

FIGS. 22A and 22B depict a shaft subassembly 800 in accordance with some embodiments. The shaft subassembly 800 forms a portion of the third rotary joint 246 and includes a third rotary shaft 247 corresponding to the third rotation axis 248. The shaft subassembly includes support brackets 810a, 810b. In some embodiments, the third rotary joint shaft 247 is separated from the support brackets 810a, 810b, by bearings 812a, 812b.

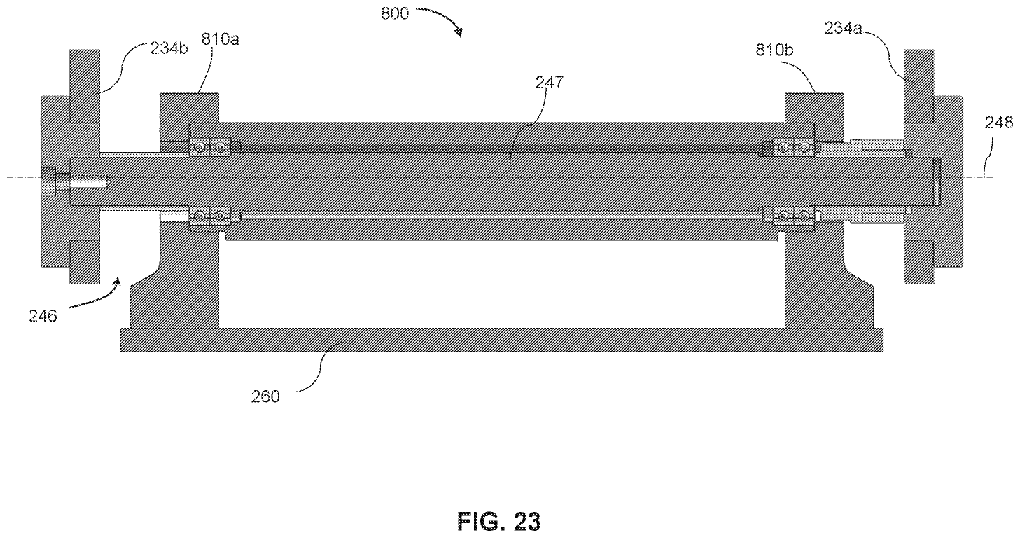

FIG. 23 is a cross-sectional view of the shaft subassembly 800 rotationally coupled to the distal ends 234a and 234b of the parallel linkage side plates 230a and 230b, and attached to the mounting 260 of the positioning arm 240 via the support brackets 810a, 810b in accordance with some embodiments. The distal ends 234a and 234b of the parallel linkage side plates 230a and 230b are rotationally locked to the third rotary shaft 247 and are rotationally coupled to the mounting 260 of the positioning arm 240 via the third rotary joint 246 and the support brackets 810a, 810b.

FIGS. 24A and 24B depict another example pitch system 200′ in accordance with some embodiments. An angled linkage body 220′ of pitch system 200′ includes side plates connected with each other. Pitch system also includes a central parallel linkage body 230′ in accordance with some embodiments. In the pitch system 200′, a single central angled rigid member 225′ is pivotably connected with the central parallel linkage body 230′. In the pitch system 200′, two side parallel rigid members 235′ are employed. A middle portion of each side parallel rigid members 235′ is depicted with a dotted line for illustrative purposes.

In some embodiments, the pitch system may also include a third link stage including at least one third linkage body and at least one third rigid member. In some embodiments, the third link stage may be employed instead of a positioning arm. In some embodiments, the pitch system can include a single central angled rigid member and a single central parallel rigid member, as further described below.

### Pitch System Having A Single Angled Rigid Member and A Single Parallel Rigid Member

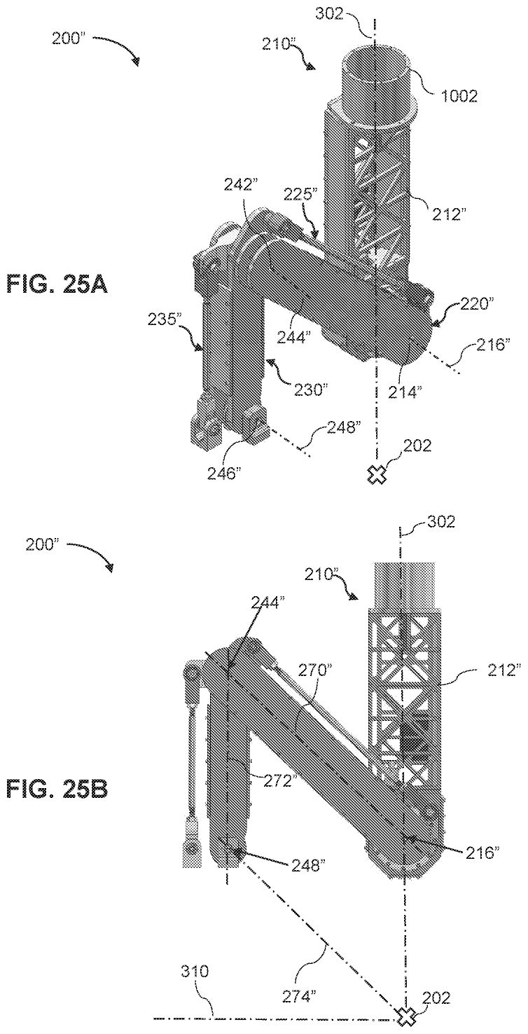

FIG. 25A is a perspective view of another example pitch system 200″ in accordance with some embodiments. The pitch system 200″ controls pitch angle motion about the virtual center 202. The virtual center 202 can be defined as an intersection point at an intersection of the yaw axis 302 of the yaw system 300 (as also illustrated in FIG. 6) and a pitch axis (not shown). The pitch axis is a transverse or lateral axis passing through the pitch system 200″. Rotation about the pitch axis is called pitch. Pitch changes the vertical direction that the insertion axis 304 is pointing. The pitch axis can pass through the virtual center 202. The pitch axis provides a reference allowing the pitching system 200″ to pitch up and down the insertion axis 304 (as also illustrated in FIG. 6) relative to the horizontal line 310 (as illustrated in FIG. 25B). In use, one or more robotic arm assemblies and/or a camera assembly supported by the positioning arm 240 (as illustrated in FIG. 6) may collectively be inserted through the trocar mount 150 as illustrated in FIG. 6) and the trocar 50 (as illustrated in FIG. 6) and into the patient along the insertion axis 304 or an axis parallel to the insertion axis 304 that also passes through the trocar 50.

The pitch system 200″ generates and controls pitch motion about the virtual center 202. This pitch motion enables systems, subsystems, units and/or other components mounted to or supported by the positioning arm 240 (as illustrated in FIG. 6), for example the robotic arm subassembly 21 shown of FIG. 4A to be pitched upward or downward relative to the virtual center 202, which usually corresponds to an incision site or a site within the trocar 50.

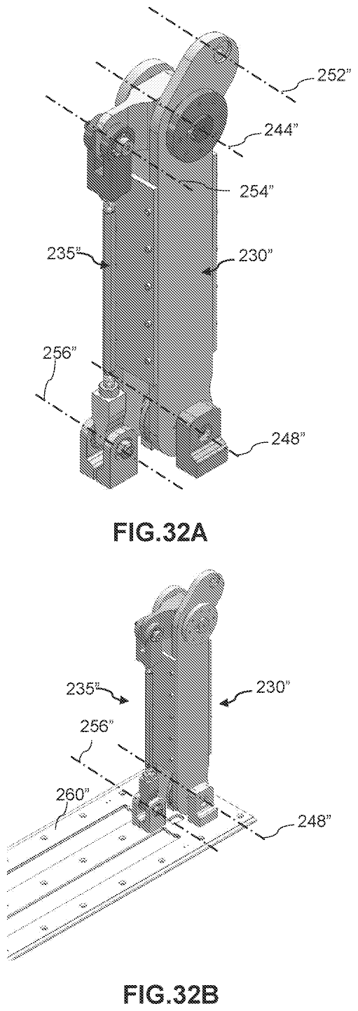

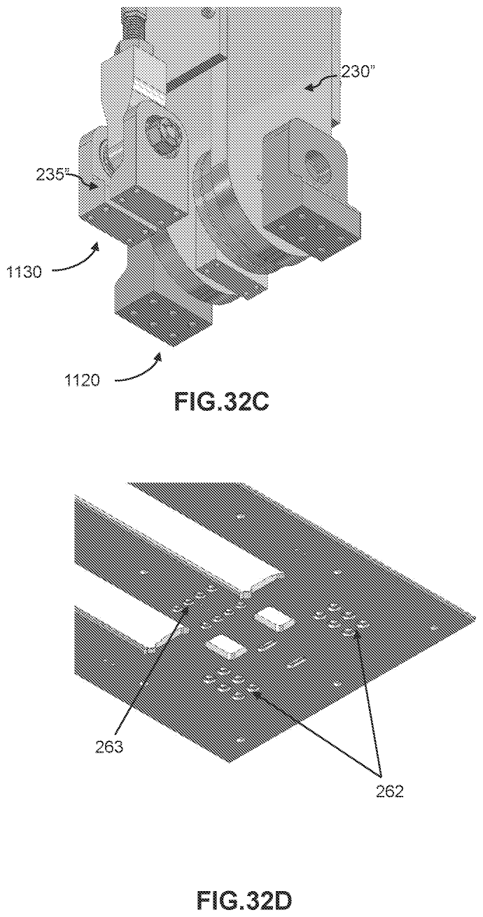

The pitch system 200″ includes a pitching housing assembly 210″ having a pitching housing 212″, an angled linkage body 220″, a single angled rigid member 225″, a parallel linkage body 230″, a single parallel rigid member 235″, and a mounting (e.g., a mounting plate) 260″ (as illustrated in FIG. 32B) for the positioning arm 240 (as illustrated in FIG. 6). The pitching housing assembly 210″ has a first end 1002 configured to be connected with, configured to be connected to, connected with, or connected to the yaw system 300 (as illustrated in FIG. 6) that defines the yaw axis 302. The pitch system 200″ also includes a first rotary joint 214″ having a first rotation axis 216″, which may be included in the pitch housing assembly 210″, a second rotary joint 242″ having a second rotation axis 244″, and a third rotary joint 246″ having a third rotation axis 248″.

The angled linkage body 220″ has a proximal end rotationally coupled to the pitch housing assembly 210″ via the first rotary joint 214″, and has a distal end rotationally coupled to the parallel linkage body 230″, via the second rotary joint 242″. In the context of components extending between and connecting the pitch housing assembly 210″ to the positioning arm 240 (as illustrated in FIG. 6), a proximal end of a component is more closely connected to the pitch housing and a distal end of the component is further away or less directly connected to the pitch housing than the proximal end. The angled linkage body 220″ has a first linkage body axis 270″ that is defined as a line perpendicular to the second rotation axis 244″ and extending from the second rotation axis 244″ at the second rotary joint 242″ through the first rotation axis 216″ at the first rotary joint 214″ and intersecting the yaw axis 302 joint (as illustrated in FIG. 25B). The first linkage body axis 270″ is never parallel to the yaw axis 302, but is instead at a nonzero angle relative to the yaw axis 302 (as illustrated in FIG. 25B), which is why at least one linkage body that connects to a pitch housing at a first rotary joint is referred to as at least one angled linkage body herein.

The parallel linkage body 230″ has a proximal end rotationally coupled to the angled linkage body 220″ via the second rotary joint 242″, and has a distal end rotationally coupled to the positioning arm 240 (as illustrated in FIG. 6) via the third rotary joint 246″. The parallel linkage body 230″ has a second linkage body axis 272″ (as illustrated in FIG. 25B) that is defined as a line perpendicular to and extending from the third rotation axis 248″ at the third rotary joint 246″ to the second rotation axis 244″ and intersecting the first linkage body axis 270″. The second linkage body axis 272″ is always parallel to the yaw axis 302 (as illustrated in FIG. 25B), which is why at least one linkage body that connects to positioning arm or a mount for a positioning arm at the third rotary joint is referred to as at least one parallel linkage body herein.

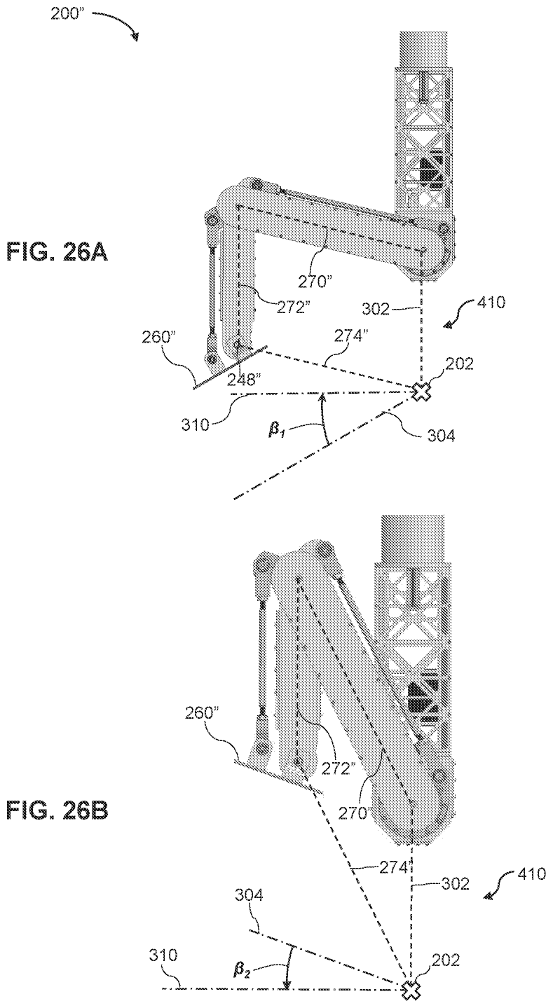

The angled rigid member 225″ is configured to cause rotation of the parallel linkage body 230″ in response to a rotation of the angled linkage body 220″. The parallel rigid member 235″ is configured to cause a rotation of the positioning arm 240 (as illustrated in FIG. 6) in response to a rotation of the parallel linkage body 230″. The angled rigid member 225″ and the parallel rigid member 235″ are also configured to constrain motion of the angled linkage body 220″, the parallel linkage body 230″, and the positioning arm 240 relative to each other and relative to the pitch housing 212″. Such configuration can maintain an orientation of the second linkage body axis 272″ parallel to the yaw axis 302 and to maintain an orientation of the first linkage body axis 270″ parallel to a line 274″ perpendicular to the third rotational axis 248″ extending from the virtual center 202 to the third rotational axis 248″ (also illustrated in FIGS. 25A and 25B), during rotation of the angled linkage body 220″ relative to the pitch housing 212″. Further explanation of how the rigid members constrain motion is provided below with respect to FIG. 25B. Further explanation of the rigid members is provided below with respect to FIGS. 26A and 26B.

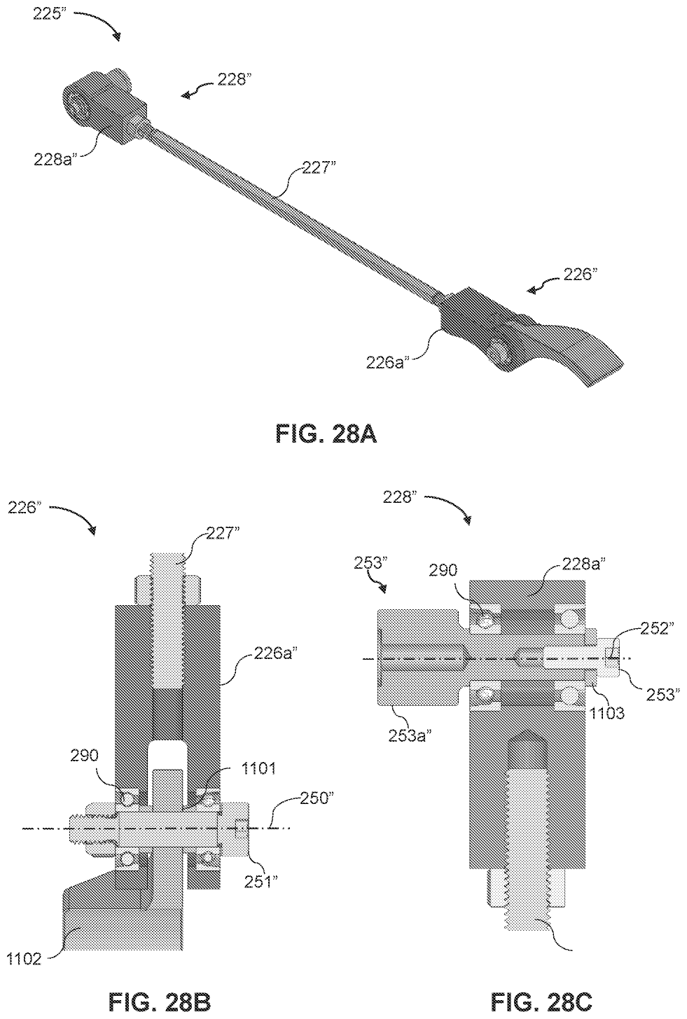

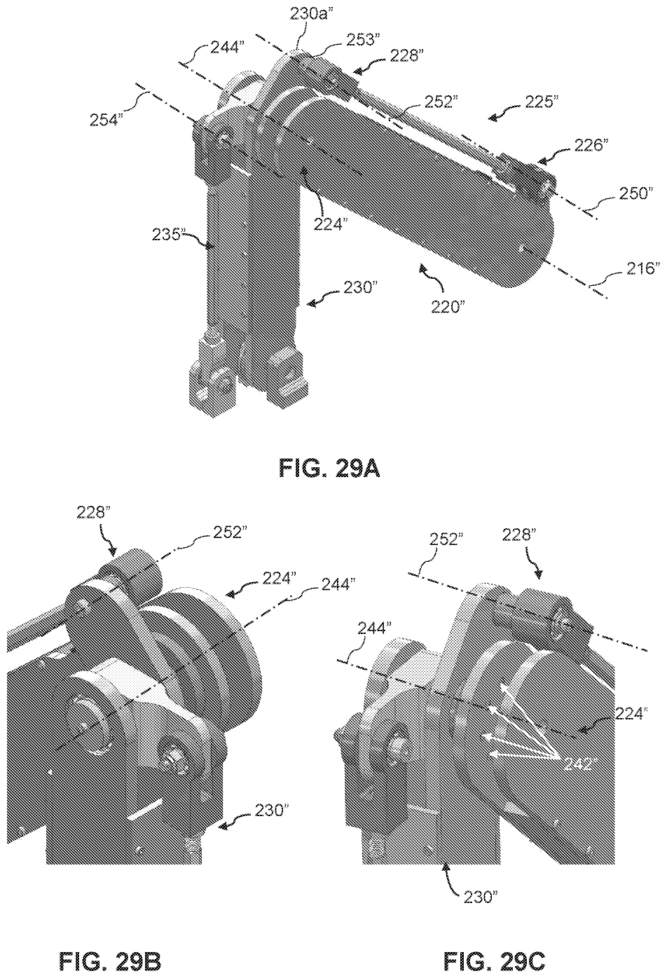

FIG. 25B is a side view of the pitch system 200″ in accordance with some embodiments. When in use, the pitch housing assembly 210″, the angled rigid member 255″, and the parallel rigid member 235″ are configured to constrain motion of the parallel linkage body 230″, and the positioning arm 240 or the mount 260″ (as illustrated in FIG. 32B) for the positioning arm 240 to maintain an orientation of the second linkage body axis 272″ parallel to the yaw axis 302 and to maintain an orientation of the first linkage body axis 270″ parallel to the line 274″ perpendicular to the third rotational axis 248″ extending from the virtual center 202 to the third rotational axis 248 during rotation of the angled linkage body 220″ relative to the pitch housing 212″. For example, when in use, the angled linkage body 220″ can be rotated relative to the pitch housing 212″ about the first rotation axis 216″. Due to this rotation of the angled linkage body 220″ with respect to the pitch housing 212″ and its rotational coupling to the parallel linkage body 230″, this also causes the angled rigid member 225″, which is rotationally connected to the pitch housing 212″ and the parallel linkage body 230″, to rotate relative to the pitch housing 212″ at a first pivot axis 250″ (as illustrated in FIGS. 28B and 29A) and to rotate relative to the first and second parallel linkage body 230″ at a second pivot axis 252″ (as illustrated in FIGS. 28C and 29A-29C). Assuming a particular yaw orientation, the at least one parallel linkage body 230″ can be translated in the X-Y plane of FIG. 25B while remaining parallel to the yaw axis 302. Movements of the angled linkage body 220″ and the parallel linkage body 230″ are constrained to form a parallelogram shape created by the first linkage body axis 270″, the second linkage body axis 272″, a line 274″ perpendicular to the third rotational axis 248″ and extending from the virtual center 202 to the third rotational axis 248″, and the yaw axis 302″. Example movements of the pitch system 200″ to pitch up and down the insertion axis 304 relative to the horizontal line 310 constrained by a respective parallelogram are further described with respect to FIGS. 26A and 26B.

FIG. 26A is a side view of the pitch system 200″ adjusted to pitch up the insertion axis 304 for an angle of positive 30 degrees relative to the horizontal line 310 passing through the virtual center 202 in accordance with some embodiments. As illustrated in FIG. 26A, the pitch system 200″ can be adjusted to pitch up the insertion axis 304 to form a first angle β₁ relative to the horizontal line 310. As depicted, the pitch angle β₁ is about +30°.

FIG. 26B is a side view of the pitch system 200″ adjusted to pitch down the insertion axis 304 for an angle of negative 20 degrees relative to the horizontal line 310 passing through the virtual center 202 in accordance with some embodiments. The pitch system 200″ can be adjusted to pitch down the insertion axis 304 to form a second angle β₂ to the horizontal line 310. As depicted, the pitch angle α₁ is about −20°. Notably, the first and second angles are not limited to the examples provided herein. For illustrative purposes, portions of the first linkage body axis 270″, the second linkage body axis 272″, the line 274″, and the yaw axis 302 that form a parallelogram shape are illustrated in FIGS. 26A and 26B. As shown in FIGS. 26A and 26B, the first linkage body axis 270″, the second linkage body axis 272″, the line 274″, and the yaw axis 302 form a parallelogram 410 that maintains a constant position of the virtual center 202 for different orientations of the insertion axis 304, but whose shape changes depending on an orientation of the first linkage body axis 270″ relative to the yaw axis 302.

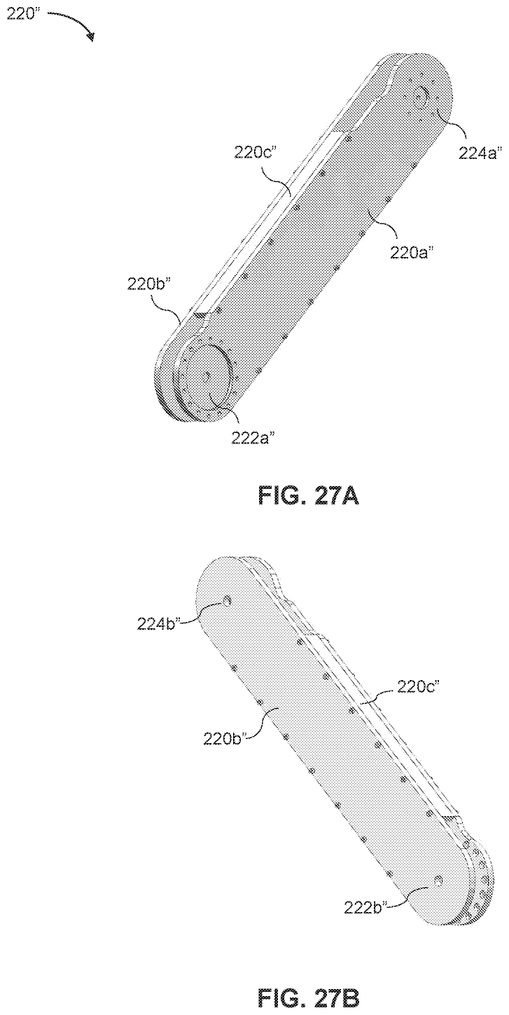

FIG. 27A is a perspective view from right side of the angled linkage body 220″ in accordance with some embodiments. FIG. 27B is a perspective view from left side of the angled linkage body 220″ in accordance with some embodiments. The angled linkage body 220″ includes an inner angled linkage side plate 220a″, an outer angled linkage side plate 220b″ and a top angled linkage plate 220c″. The inner and outer angled linkage side plates 220a″ and 220b″ each have a proximal end 222a″ and 222b″ rotationally coupled to the pitch housing 212 at the first rotary joint 214″ (e.g., as illustrated in FIG. 25A), and each have a distal end 224a″ and 224b″ rotationally coupled to the parallel linkage body 230″ at the second rotary joint 242″ (e.g., as illustrated in FIGS. 25A and 25B). The top angled linkage plate 220c″ connects the inner angled linkage side plate 220a″ with the outer angled linkage side plate 220b″ such that the inner angled linkage side plate 220a″, the outer angled linkage side plate 220b″ and the top angled linkage plate 220c″ forms a single-piece angled linkage body.

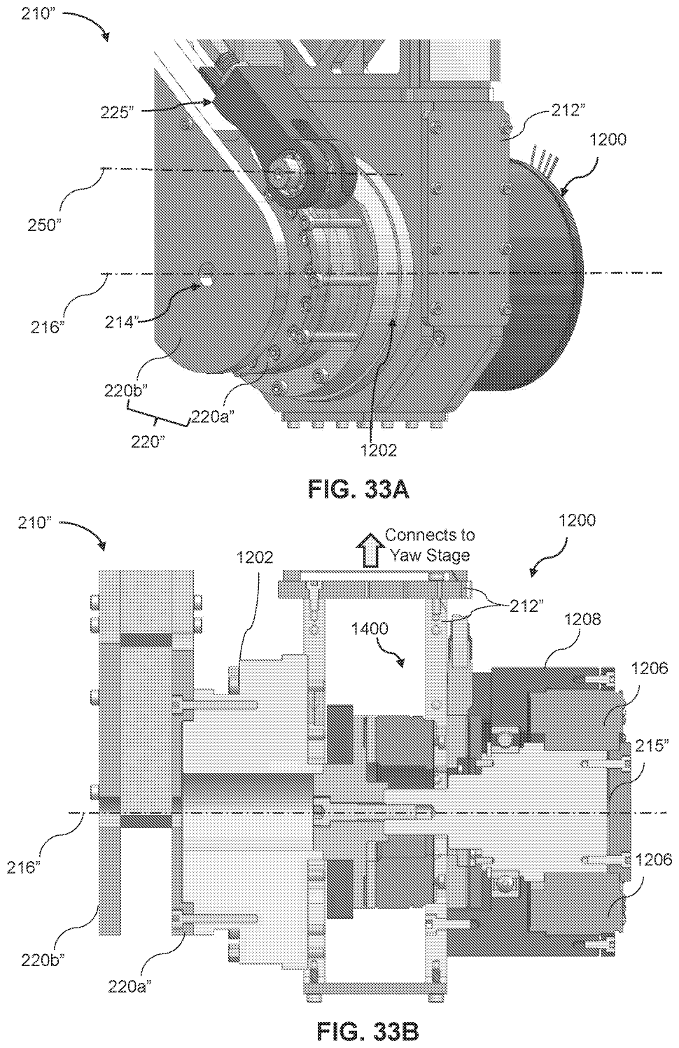

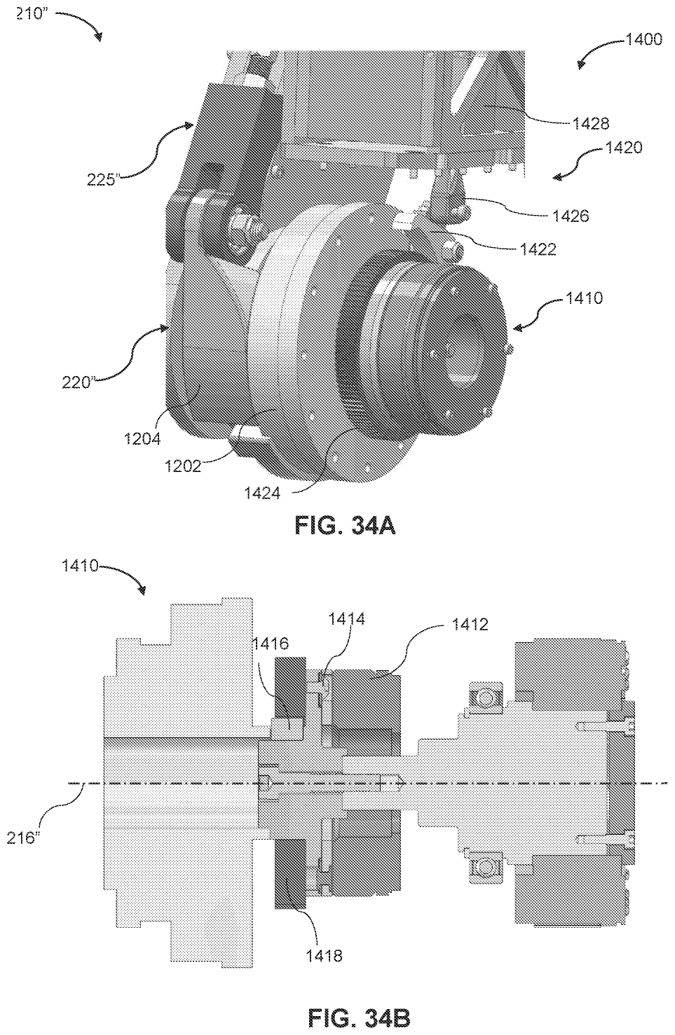

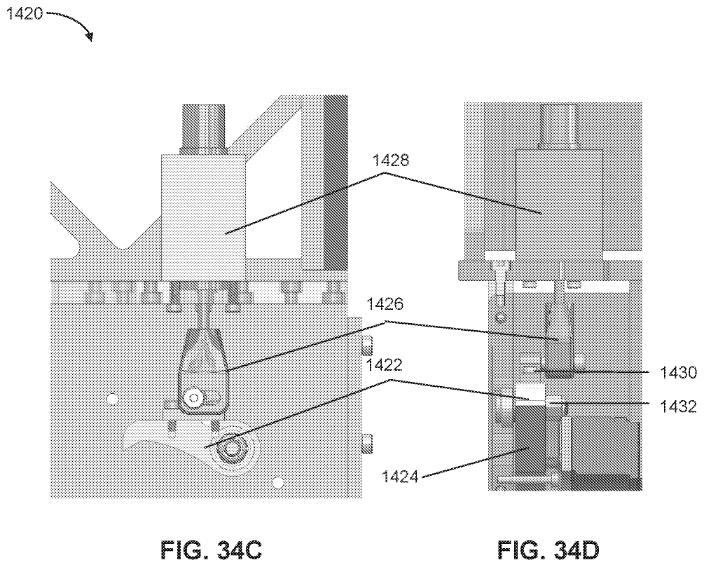

FIG. 28A is a perspective view of the angled rigid member 225″ in accordance with some embodiments. The angled rigid member 225″ can cause a parallel linkage body rotation of the parallel linkage body 230″ relative to the angled linkage body 220″ about the second rotary joint 242″ (e.g., as illustrated in FIGS. 25A and 25B) due to the angled linkage body rotation of the angled linkage body 220″. The angled rigid member 225″ has a proximal end 226″ rotationally coupled to the pitch housing 212 at a first pivot axis 250″ (as illustrated in FIGS. 28B and 29A) offset from the first rotation axis 216″. The angled rigid member 225″ has a distal end 228″ rotationally coupled to a proximal end 232″ of the parallel linkage body 230″ at a second pivot axis 252″ parallel to and offset from the second rotation axis 244″, as further described with respect to FIG. 29A. The angled rigid member 225″ also includes an elongated center portion 227″ (e.g., a turnbuckle rod, a shaft or the like) that is threadedly attached at each end to an end portion 226a″ and 228a″. A tension or a length of the angled rigid member 225″ can be adjustable via rotation of the center portion 227″ of the turnbuckle relative to the end portions 226a″ and 228a″, respectively. The angled linkage body 220″ are not directly connected to the angled rigid member 225″, but both are connected to the pitch housing 212″, as further described with respect to FIGS. 33A and 34A.

FIG. 28B is a cross-sectional view of the proximal end 226″ of angled rigid member 225″ in accordance with some embodiments. The proximal end 226″ is coupled to a gear standoff plate 1204 of the pitch housing assembly 210″, as further described with respect to FIGS. 33A and 34A. One end of the end portion 226a″ is coupled to the elongated center portion 227″, and the other end of the end portion 226a″ is coupled to the gear standoff plate 1204 via an axle shaft 251″ having a first pivot axis 250″. In some embodiments, the axle shaft 251″ is part of a locking bolt. Bearings 290 separate the proximal end 226″ of the angled rigid member 225″ from the axle shaft 251″ having the first pivot axis 250″. A turnbuckle bearing spacer 1101 is located between the bearings 290 and the gear standoff plate 1102.

FIG. 28C is a cross-sectional view of the distal end 228″ of the angled rigid member 225″ in accordance with some embodiments. The distal end 228″ is rotationally coupled to a proximal end of the parallel linkage body 230″ at a second pivot axis 252″. The bearings 290 separate the angled rigid member 225″ from an axle shaft 253″ having the second pivot axis 252″. As illustrated, the axle shaft 253″ include two bolts 253a″ and 253b″. A bearing cap plate 1103 is located between the bearings 290 and the bolt 253b″. In some embodiments, the axle shaft 253″ is formed, in part, by one or more locking bolts.

FIG. 29A is a perspective view of the angled linkage body 220″ rotationally coupled to the parallel linkage body 230″ in accordance with some embodiments. FIG. 29B is a perspective view from left side of a connection portion connecting the angled linkage body 220″ with the parallel linkage body 230″ in accordance with some embodiments. FIG. 29C is a perspective view from right side of the connection portion connecting the angled linkage body 220″ with the parallel linkage body 230″ in accordance with some embodiments.

As illustrated in FIGS. 29A-29C, the proximal end 226″ of the angled rigid member 225″ can be rotationally coupled to the pitch housing 212 (as illustrated in FIGS. 25A and 25B) at the first pivot axis 250″ parallel to and offset from the first rotation axis 216″. The distal end 228″ of the angled rigid member 225″ is rotationally coupled to the parallel linkage side plate 230a″ of the parallel linkage body 230″ at the second pivot axis 252″ parallel to and offset from the second rotation axis 244″. As illustrated, the axle shaft 253″ of the distal end 228″ is coupled to, connected to, and/or mounted to a parallel linkage side plate 230a″ of the parallel linkage body 230″. The angled linkage body 220″ is rotationally coupled to the parallel linkage body 230″ via the second rotary joint 242″ having the second rotation axis 244″. The parallel rigid member 235″ can be rotationally coupled to the parallel linkage body 230″ at the third pivot axis 254″. The parallel rigid member 235″ can be rotationally connected to the angled linkage body 220″ indirectly through one or more elements or components rotationally locked to the angled linkage body 220″ at the third pivot axis 254″ parallel to and offset from the second rotation axis 244″.

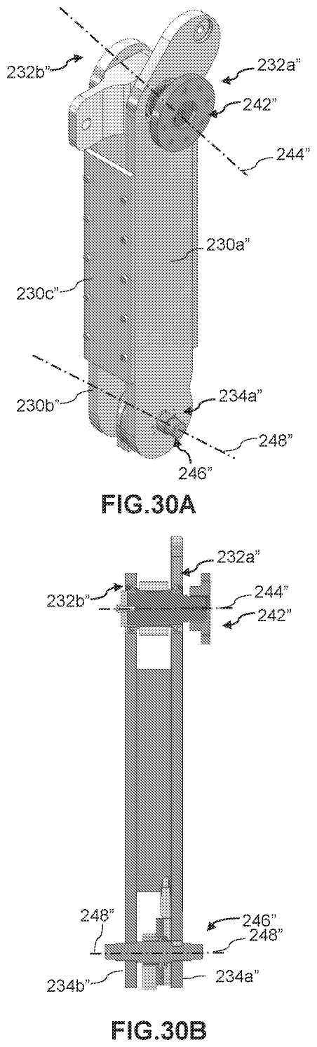

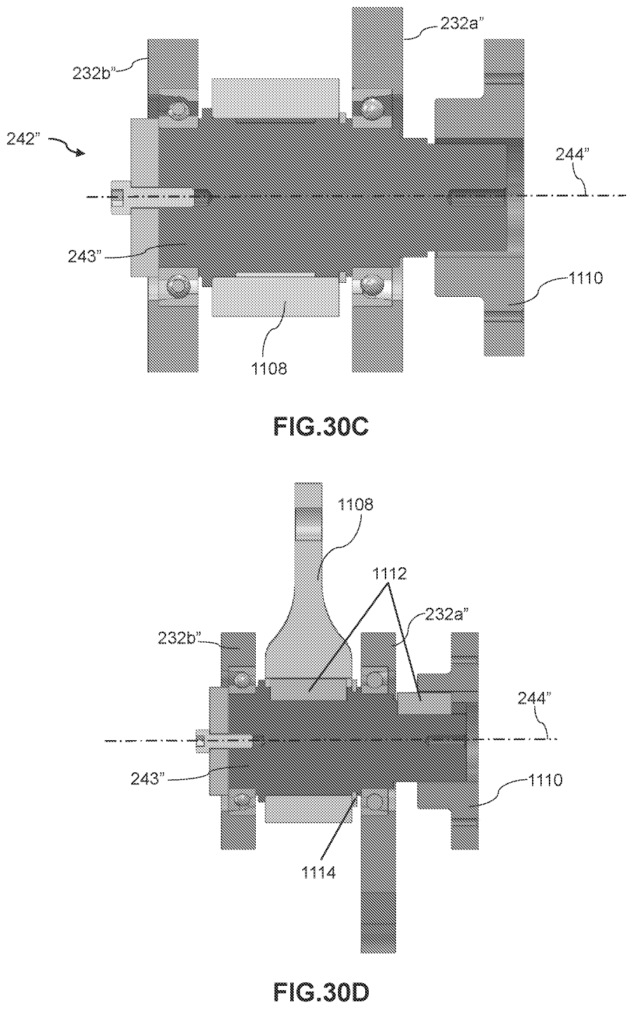

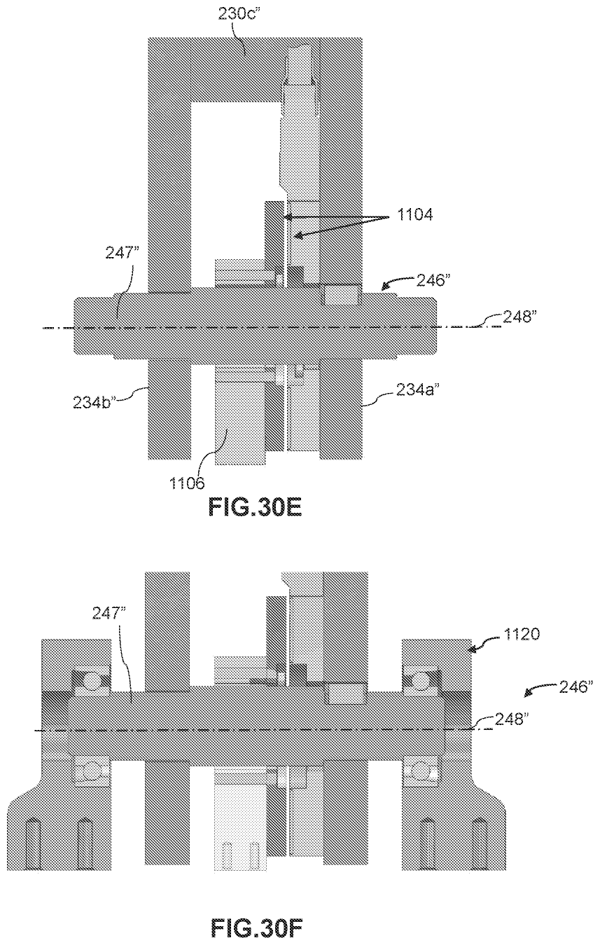

FIG. 30A is a perspective view of the parallel linkage body 230″ rotationally coupled to the second rotary joint 242″ and the third rotary joint 246″ in accordance with some embodiments. FIG. 30B is a cross-sectional view of the parallel linkage body 230″ rotationally coupled to the second rotary joint 242″ and the third rotary joint 246″ in accordance with some embodiments. The parallel linkage body 230″ includes the first parallel linkage side plate 230a″, the second parallel linkage side plate 230b″, and an additional plate 230c″ that may connect the first and second parallel linkage side plates 230a″, 230b″, for example, to provide any of strength, rigidity, and support an/or to reduce torsion. Each of the first and second parallel linkage side plates 230a″ and 230b″ has a proximal end 232a″ and 232b″. The proximal end 232a″ of the first parallel linkage side plate 230a″ can be rotationally coupled to the distal end 224″ of the angled linkage body 220″ via the second rotary joint 242″ having the second rotation axis 244″. The proximal end 232b″ of the second parallel linkage side plate 230b″ can be rotationally coupled to the second rotary joint 242″ having the second rotation axis 244″. Each of the first and second parallel linkage side plates 230a″ and 230b″ has a distal end 234a″ and 234b″ rotationally coupled to the positioning arm 240 (as illustrated in FIG. 6) directly or via a mounting (e.g., a mounting plate) 260″ (as illustrated in FIG. 32B) via a third rotary joint 246″ having the third rotation axis 248″. The third rotation axis 248″ can be parallel to the first rotation axis 216″.

FIG. 30C is a rear cross-sectional view of the second rotary joint 242″ coupled to the proximal end 232a″ and 232b″ of the first and second parallel linkage side plates 230a″, 230b″ in accordance with some embodiments. FIG. 30D is a bottom cross-sectional view of the second rotary joint 246″ coupled to the proximal end 232a″ and 232b″ of the first and second parallel linkage side plates 230a″, 230b″ in accordance with some embodiments. The second rotary joint 242″ includes a second rotary joint shaft 243″ corresponding to the second rotation axis 244″. The second rotary joint 242″ includes a turnbuckle mount 1108 coupled to, connected to or mounted to the parallel rigid member 235″ as further described with respect to FIG. 32A. The second rotary joint 242″ includes a shaft support 1110 configured to support the second rotary shaft 247″. The shaft support 1110 is also coupled to, connected to or mounted to the distal end 224″ of the angled linkage body 220″ as described with respect to FIGS. 29A-29C. The second rotary joint 242″ includes machine keys 1112 and a retaining key 1114 to retain the movements of the turnbuckle mount 1108 and the shaft support 1110.

FIG. 30E is a cross-sectional view of the third rotary joint 246″ coupled to the distal end 234a″ and 234b″ of the first and second parallel linkage side plates 230a″, 230b″ in accordance with some embodiments. The third rotary joint 246″ includes a third rotary shaft 247″ corresponding to the third rotation axis 248″. The third rotary joint 246″ includes a joint encoder 1104 configured to detect rotation angle or linear displacement of the third rotary joint 246″. The third rotary joint 246″ includes an encoder rotor mount 1106 configured to support the joint encoder 1104.

FIG. 30F is a cross-sectional view of the third rotary joint 246″ coupled to the parallel linkage body 230″ and further coupled to a third joint bracket 1120 in accordance with some embodiments. The third rotary shaft 247″ is coupled to the third joint bracket 1120 such that the parallel linkage body 230″ can be rotationally coupled to the positioning arm 240 or to the mounting 260″ (as illustrated in FIG. 32B) which can be configured to be attached to the positioning arm 240 via the third rotary joint 246″ at the third rotation axis 248″.

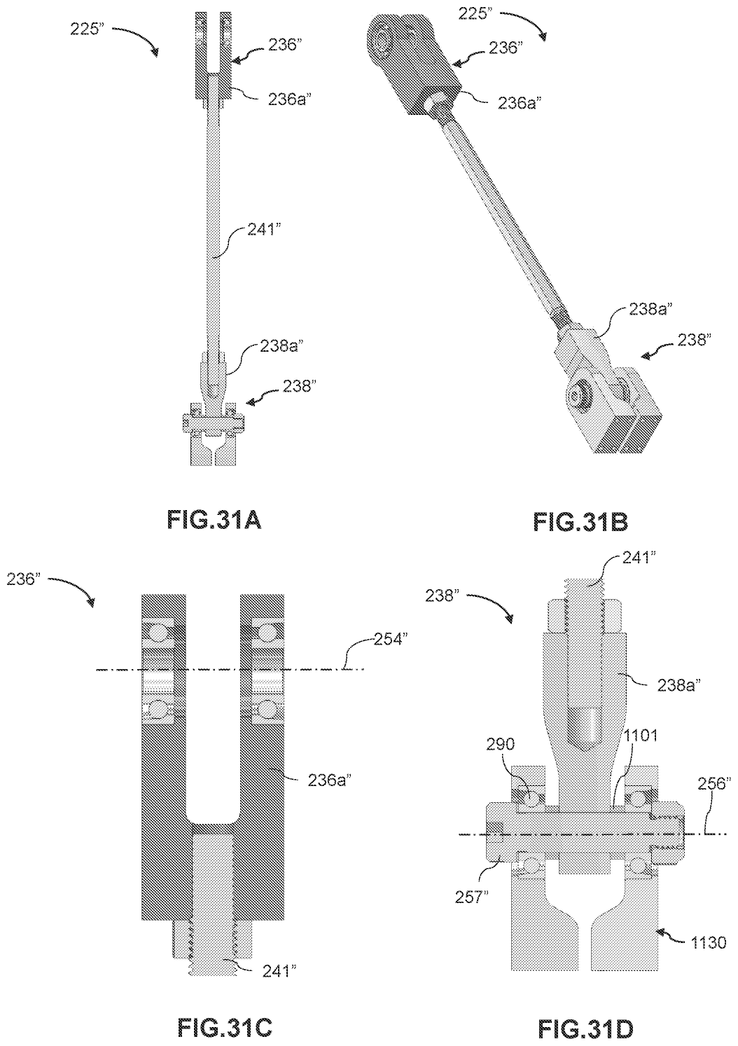

FIG. 31A is a side view of the parallel rigid member 235″ in accordance with some embodiments. FIG. 31B is a perspective view of the parallel rigid member 235″ in accordance with some embodiments The parallel rigid member 235″ can be a central rigid member. The parallel rigid member 235″ can cause a rotation of the positioning arm 240 (e.g., as illustrated in FIG. 6) relative to the parallel linkage body 230″ due to a rotation of the parallel linkage body 230″. The parallel rigid member 235″ has a proximal end 236″, an elongated center portion 241″ (e.g., a turnbuckle rod, a shaft or the like), and a distal end 238″. The elongated center portion 241″ is threadedly attached at each end to an end portion 236a″ and 238a″. A tension or a length of the parallel rigid member 235″ can be adjustable via rotation of the center portion 241″ of the turnbuckle relative to the end portions 236a″ and 238a″, respectively.

FIG. 31C is a cross-sectional view of the proximal end 236″ of the parallel rigid member 235″ in accordance with some embodiments. One end of the end portion 236a″ is coupled to the elongated center portion 241″, and the other end of the end portion 236a″ can be rotationally coupled with the angled linkage body 220″ at a third pivot axis 254″ parallel to and offset from the second rotation axis 244″ (as illustrated in FIG. 32A). The proximal end 236″ of the parallel rigid member 235″ can be rotationally connected to the angled linkage body 220″ indirectly through one or more elements or components rotationally locked to the angled linkage body 220″.

FIG. 31D is a cross-sectional view of the distal end 238″ of the parallel rigid member 235″ in accordance with some embodiments. The distal end 238″ is rotationally coupled to the positioning arm 240 or to a mounting (e.g., mounting plate) 260″ configured to be attached to the positioning arm 240 at a fourth pivot axis 256″ offset from and parallel to the third rotation axis 248″ (as illustrated in FIG. 32A). One end of the end portion 238a″ is coupled to the elongated center portion 241″, and the other end of the end portion 238a″ is rotationally coupled with a mounting bracket 1130 via an axle shaft 257″ having the fourth pivot axis 256″. The axle shaft 257″ is formed, in part, by one or more locking bolts. The bearings 290 separate the axle shaft 257″ from the mounting bracket 1130 and the turnbuckle bearing spacer 1101 is located between the bearings 290 and the end portion 238a″.

FIG. 32A is a perspective view of the parallel linkage body 230″ coupled to the parallel rigid member 235″ in accordance with some embodiments. FIG. 32B is a perspective view of the parallel linkage body 230″ and the parallel rigid member 235″ coupled to the mounting plate 260″ in accordance with some embodiments.

As illustrated in FIGS. 32A and 32B, the parallel linkage body 230″ can be rotationally coupled to the mounting plate 260″ configured to be attached to the positioning arm 240 at the third rotation axis 248″. The parallel rigid member 235″ can be rotationally coupled to the mounting plate 260″ at the fourth pivot axis 256″ offset from and parallel to the third rotation axis 248″.

FIG. 32C is a perspective view from bottom of the parallel linkage body 230″ coupled to the parallel rigid member 235″ and further coupled to the third joint bracket 1120 in accordance with some embodiments. FIG. 32D is a perspective view from bottom of the mounting plate 260″ accordance with some embodiments. The third joint bracket 1120 can be threadedly attached to holes or threaded holes 262 of the mounting plate 260. The mounting bracket 1130 can be threadedly attached to holes or threaded holes 263 of the mounting plate 260 such that the parallel linkage body 230″ and the parallel rigid member 235″ can be coupled to the mounting plate 260″.

FIG. 33A is a perspective view from the left side of the pitch housing assembly 210″ having an actuator assembly 1200 connecting to the angled linkage body 220″ and the angled rigid member 225″ in accordance with some embodiments. The actuator assembly 1200 can provide a direct drive and provide several advantages including increased stiffness by elimination of the belts, which can simplify the feedback control. The actuator assembly 1200 can also include a permanent magnet brake that has a lighter weight (with lower torsional capacity and power consumption) due to speed reduction of the strain wave gearhead. The pitch housing assembly 210″ includes the pitch housing 212″ that supports various components, the first rotary joint 214″, and the actuator assembly 1200 configured to drive a rotation of the angled linkage body 220″ relative to the pitch housing 212″ about the first rotary joint 214″ to cause an angled linkage body rotation. The inner angled linkage side plate 220a″ of the angled linkage body 220″ is rotationally coupled to an actuator 1202 of the actuator assembly 1200 via the first rotary joint 214″ having the first rotation axis 216″. The angled rigid member 225″ is rotationally coupled to the actuator drive 1202 at the first pivot axis 250″. The actuator assembly 1200 is further described with respect to FIG. 33B.

FIG. 33B is a cross-sectional view of the pitch housing assembly 210″ in FIG. 33A in accordance with some embodiments. The actuator assembly 1200 includes the actuator drive 1202 (e.g., a strain wave gearhead or other suitable gearhead, or the like), one or more actuators 1206 (e.g., motors or other suitable actuators), an actuator housing 1208, a braking system 1400, and other related hardware. The one or more actuators 1206 can be rotationally coupled (e.g., rotationally locked) to a first rotary joint shaft 215″ of the first rotary joint 214″. The first rotary joint shaft 215″ is rotationally coupled (e.g., rotationally locked) to the proximal end of the angled linkage body 220″. The braking system 1400 is configured for braking of the first rotary joint shaft 215″ of the first rotary joint 214″ relative to the pitch housing 212″. The braking system 1400 is further described with respect to FIGS. 35A-35D.

FIG. 34A is a perspective view from the right side of the pitch housing assembly 210″ of FIG. 33A illustrating a braking system 1400. The braking system 1400 can be coaxial with the actuator drive 1202 that is rotationally coupled to the angled linkage 220″ and the angled rigid member 225″ via a standoff plate 1204 (e.g., a gearhead standoff plate). If the braking system 1400 has enough torque capacity to counter act the static load of the pitch system 200″, it prevents all rotation. The braking system 1400 can include a primary brake 1410 (e.g., permanent magnet or the like) that can be coaxial with an output axis of the actuator 1200, as further described with respect to FIG. 34B. The braking system 1400 can further include a secondary brake 1420, as further described with respect to FIGS. 34C and 34D.

FIG. 34B is a cross-sectional view of the primary brake 1410 of FIG. 34A in accordance with some embodiments. Some components of the pitch housing assembly 210″ are omitted for illustration purpose. The primary brake 1410 includes a brake rotor 1414 fixed to the first joint rotary shaft 215″ of the first rotary joint 214″ and a brake stator 1412 fixed to the pitch housing 121″. The primary brake 1410 further includes one or more machine keys 1416 and a ratchet gear 1418. In some embodiments, the primary brake 1410 can only include the brake rotor and the brake stator.

FIG. 34C is a side view of the secondary brake 1420 in accordance with some embodiments. FIG. 34D is a front view of the secondary brake 1420 in accordance with some embodiments. As illustrated in FIG. 34A, the secondary brake 1420 includes a pawl 1422, a ratchet gear 1424, a solenoid actuator 1428 (e.g., a linear solenoid with return spring or the like) having an adaptor 1426. The pawl 1422 can be actuated linearly or rotationally about its intended pivot point. In the case of linear actuation, a slotted pawl mount 1430 and a shoulder bolt 1432 can be used to create rotation of the pawl 1422 about its pivot point through linear actuation. The actuation can be generated electromechanically (e.g., via a solenoid actuator 1428). When the electromechanical actuator 1428 is powered off, gravity and/or a return spring can be employed to engage the pawl 1422 with the ratchet gear 1424, preventing rotation of the pitch actuator assembly 1200. When the electromechanical actuator 1428 is powered on, the force or torque generated by the actuator 1428 overcomes the forces of gravity and/or return spring to disengage the pawl 1422 from the ratchet gear 1424, permitting rotation of the actuator assembly 1200. For example, when the solenoid actuator 1428 is energized, the solenoid 1428 pulls the adapter 1426 upward, rotating the pawl 1424 and disengaging it from the ratchet gear 1424.

While embodiments of the present disclosure have been shown and described herein, it will be obvious to those skilled in the art that such embodiments are provided by way of example only. Numerous variations, changes, and substitutions will now occur to those skilled in the art without departing from the invention. It may be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A pitch system for controlling a tilt orientation of one or more surgical instruments relative to a virtual center, the pitch system comprising:
at least one angled linkage body having a proximal end, a distal end, and a first linkage body axis at an acute angle with respect to a yaw axis of the pitch system during use;
at least one parallel linkage body having a proximal end, a distal end, and a second linkage body axis parallel to the yaw axis of the pitch system during use;
a pitch housing assembly having a first end configured to be connected with, configured to be connected to, connected with, or connected to a yaw system defining the yaw axis, the pitch housing assembly comprising:
a pitch housing;
a first rotary joint having a first rotational axis perpendicular to and intersecting the yaw axis; and
an actuator assembly configured to drive a rotation of the at least one angled linkage body relative to the pitch housing about the first rotary joint causing an angled linkage body rotation;
a second rotary joint having a second rotation axis parallel to the first rotation axis, the proximal end of the at least one angled linkage body rotationally coupled to the pitch housing at the first rotary joint and a proximal end of the at least one parallel linkage body rotationally coupled to the at least one angled linkage body at the second rotary joint;
a third rotary joint having a third rotation axis parallel to the first rotation axis;
at least one angled rigid member configured to cause a parallel linkage body rotation of the at least one parallel linkage body relative to the at least one angled linkage body about the second rotary joint due to the angled linkage body rotation, the proximal end of the at least one angled rigid member rotationally coupled to the pitch housing at a first pivot axis parallel to and offset from the first rotation axis, and a distal end of the at least one angled rigid member rotationally coupled to the at least one parallel linkage body at a second pivot axis parallel to and offset from the second rotation axis;
the proximal end of the at least one parallel linkage body rotationally coupled to the distal end of the at least one angled linkage body at the second rotary joint, and the distal end of the at least one parallel linkage body rotationally coupled to a positioning arm or a mounting for a positioning arm at the third rotary joint; and
at least one parallel rigid member configured to cause a positioning arm rotation of the positioning arm relative to the at least one parallel linkage body about the third rotary joint due to the parallel linkage body rotation, a proximal end of the at least one parallel rigid member rotationally coupled with the at least one angled linkage body at a third pivot axis parallel to and offset from the second rotary joint, a distal end of the at least one parallel rigid member rotationally coupled with the positioning arm or the mounting for the positioning arm at a fourth pivot axis offset from and parallel to the third rotary joint;
wherein, when in use, an intersection point at an intersection of the yaw axis and a pitch axis is the virtual center; and
wherein, when in use, the pitch housing assembly, the at least one angled rigid member, and the at least one parallel rigid member are configured to constrain motion of the at least one angled linkage body, the at least one parallel linkage body, and the mounting or the positioning arm to maintain an orientation of the second linkage body axis parallel to the yaw axis and to maintain an orientation of the first linkage body axis body parallel to a line perpendicular to the third rotational axis extending from the virtual center to the third rotational axis during rotation of the at least one angled linkage body relative to the pitch housing.

2. The pitch system of claim 1, wherein the orientation of the first linkage body axis is defined as an orientation of a first line perpendicular to the second rotation axis and extending from the second rotation axis at the second rotary joint though the first rotations axis and intersecting the yaw axis; and
wherein the orientation of the at least second linkage body axis is defined as an orientation of a second line perpendicular to and extending from the third rotation axis at the third rotary joint to the second rotation axis and intersecting with the first line.

3. The pitch system of claim 1, wherein the at least one angled linkage body comprises a first angled linkage side plate and a second angled linkage side plate.

4. The pitch system of claim 1, wherein the at least one angled rigid member comprises a first side angled rigid member and a second side angled rigid member.

5. The pitch system of claim 1, wherein the at least one parallel linkage body comprises a first parallel linkage side plate and a second parallel linkage side plate.

6. The pitch system of claim 1, wherein the at least one parallel rigid member includes a central rigid member.

7. The pitch system of claim 1, wherein the at least one parallel linkage body comprises a first parallel linkage side plate and a second parallel linkage side plate each having a proximal end and a distal end; and
wherein the at least one angled rigid member comprises a first side angled rigid member and a second side angled rigid member each having a proximal end and a distal end, the distal end of the first side angled rigid member rotationally connected to the proximal end of the first parallel linkage side plate at the second pivot axis, and the distal end of the second side angled rigid member rotationally connected to the proximal end of the second parallel linkage side plate at the second pivot axis.

8. The pitch system of claim 7, wherein the at least one angled linkage body comprises a first angled linkage side plate and a second angled linkage side plate each having a proximal end and a distal end; and
wherein the second rotary joint includes a second rotary joint shaft rotationally locked to the at least one angled linkage body; and
wherein the at least one parallel rigid member includes a central parallel rigid member having a proximal end and a distal end; and
wherein the pitch system further comprises:
a first mounting bracket including a first axle shaft, the first mounting bracket attached to and rotationally locked to the second rotary joint shaft, the first mounting bracket rotatably connecting with the proximal end of the central rigid member at the third pivot axis via the first axle shaft; and
a second mounting bracket including a second axle shaft, the second mounting bracket affixed to or connected to the mounting for the positioning arm or to the positioning arm and rotatably connected with the distal end of the central rigid member at the fourth pivot axis via the second axle shaft.

9. The pitch system of claim 1, wherein the actuator assembly comprises:
at least one actuator subassembly configured to drive an output rotation about a drive axis relative to the pitch housing; and
at least one coupling configured to couple a rotation of the at least one angled linkage side plate about the first rotary joint with the output rotation about the drive axis.

10. The pitch system of claim 9, wherein the at least one actuator subassembly comprises a motor pulley.

11. The pitch system of claim 10, wherein the motor pulley includes a motor, an encoder and a gearhead.

12. The pitch system of claim 10, wherein the actuator assembly further comprises at least one output pulley rotationally locked to a first rotary joint shaft of the first rotary joint.

13. The pitch system of claim 12, wherein the at least one coupling comprises at least one drive tape affixed to the motor pulley and to the output pulley.

14. The pitch system of claim 9, wherein the at least one actuator subassembly comprises a motor, an encoder, and a gearhead.

15. The pitch system of claim 9, further comprising a braking system configured for braking of the first rotary joint relative to the pitch housing.

16. The pitch system of claim 15, wherein the braking system comprises a brake rotor fixed to the first rotary joint shaft of the first rotary joint and a brake stator fixed to the pitch housing.

17. The pitch system of claim 15, further comprising a secondary braking system comprising a pawl and a ratchet gear configured to prevent the positioning arm from rotating in at least one direction of rotation.

18. The pitch system of claim 17, wherein the pawl is configured to disengage from the ratchet gear when power is supplied to a solenoid actuator, and wherein the pawl is configured to reengage with the ratchet gear via a spring when power is removed from the solenoid actuator.

19. The pitch system of claim 1, wherein the first rotary joint comprises a first rotary shaft rotationally locked to the at least one angled linkage body; and
wherein the pitch housing comprises a first pitched housing side plate and a second pitched housing side plate.

20. The pitch system of claim 1, wherein the pitch housing assembly further comprises one or more springs configured to offset torsional moment due to components supported by the at least one angled body member.

21. The pitch system of claim 1, wherein the second rotary joint comprises a second rotary shaft coupled to the at least one parallel linkage body; and
wherein rotation of the second rotary shaft is locked to the at least angled linkage body.

22. The pitch system of claim 1, wherein the third rotary joint comprises a third rotary shaft coupled to the positioning arm or the mounting of the positioning arm, wherein rotation of the third rotary shaft is locked to the second rotary shaft via the at least one parallel linkage body.

23. The pitch system of claim 1, wherein the third rotary joint comprises a third rotary shaft coupled to the at least one parallel linkage body, and rotatably connected to the positioning arm or the mounting of the positioning arm, wherein rotation of the positioning arm about the third rotary axis is locked to the second rotary shaft via the at least one parallel linkage body.

24. The pitch system of claim 1, wherein the first rotary joint, the second rotary joint, or the third rotary joint comprises one or more shielded ball bearings, one or more preloaded bearings, or one or more bearings in a back-to-back arrangement.

25. The pitch system of claim 1, wherein the pitch axis extends normal to the insertion axis of the positioning arm and intersects the cannula axis of a trocar.
